(19) United States
(12) Patent Application Publication
(10) Pub. No.: US 2008/0217580 A1
(43) Pub. Date: Sep. 11, 2008

(54) Organic Luminescent Element

(76) Inventors: Junichi Tanabe, Hyogo (JP); Hiroshi Yamamoto, Hyogo (JP); Norihisa Dan, Kyoto (JP)

Correspondence Address: JoAnn Villamizar;Ciba Corporation/Patent Department, 540 White Plains Road, P.O. Box 2005, Tarrytown, NY, 10591, US

(21) Appl. No.: 11/578,596

(22) PCT Filed: Apr. 11, 2005
(86) PCT No.: PCT/EP2005/051579

§ 371 Date: Oct. 16, 2006

(30) Foreign Application Priority Data
Apr. 19, 2004 (EP) 04101605.6

(51) Int. Cl. C09K 11/06; C07C 229/02; C07D 307/91; C07D 207/08; C07D 235/04; C07D 263/10; C07D 209/44; C07D 251/14

(52) U.S. Cl. 252/301.16; 560/42; 549/461; 548/539; 548/309.7; 548/239; 548/472; 544/216

(57) ABSTRACT

The present invention relates to compounds of formula (I) a process for their production and to their use for coloring high molecular weight organic materials, as fluorescent tracers, in solid dye lasers, EL lasers, in EL devices and lighting. The compounds of formula I show unique broadband luminescence in visible region.

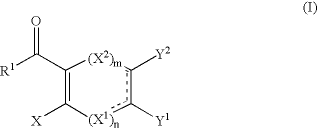

The present invention relates to compounds of formula I, a process for their production and to their use for coloring high molecular weight organic materials, as fluorescent tracers, in solid dye lasers, EL lasers, in EL devices and for lighting. The compounds of formula I show unique broadband luminescence in the visible region. On EL device comprising the compounds of formula I is, for example, suitable for lighting, as light source, monochromatic display and white luminescent backlight in LCD and color changing media (CCM) for full colour display.

Different kinds of emitting layers for obtaining white luminescence are known and examples thereof include:

(1) a product in which energy levels of respective layers of an organic EL laminated structure are specified and light is emitted using tunnel injection (EP-A-0390551);
(2) a display device using tunnel injection in the same manner as (1), an example of which is a white light emitting display device (JP-A-3-230584);
(3) a product including an emitting layer having a bi-layered structure (JP-A Nos. 2-220390 and 2-216790);
(4) a product in which an emitting layer is divided into plural layers and they are made of materials having different emitting wavelengths (JP-A-4-51491);
(5) a product having a structure in which a blue luminous body (fluorescence peak: 380 to 480 nm) and a green luminous body (480 to 580 nm) are laminated on each other, and a red fluorescent body is contained (JP-A-6-207170); and
(6) a product having a structure in which a blue luminous layer has a region containing a blue fluorescent dye and a green luminous layer has a region containing a red fluorescent dye, and further a green fluorescent body is contained (JP-A-7-142169).

As evident previous solutions for a white LED have been based in particular on the RGB approach, i.e. on mixing three colors, namely red, green and blue.

It has surprisingly been found that organic light emitting devices (OLED) showing broadband luminescence or white luminescence by a single luminescent material can be obtained, if the compounds of formula I are used as luminescent material.

Accordingly, the present invention relates to compounds of formula

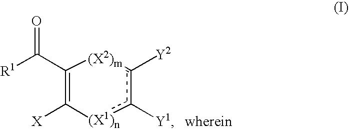

[symbol]

is a single or double bond,
n is 0, m is 1,

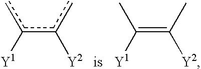

X² is —C(R²)═C(R³)—, —C(R⁴)(R⁵)—C(R⁴)(R⁵)—, —C(R²)═C(R³)—C(R⁴)(R⁵)—, —C(R⁴)(R⁵)—C(R²)═C(R³)—, —C(R⁴)(R⁵)—C(R⁴)(R⁵)—C(R²)═C(R³)—, —C(R²)═C(R³)—C(R⁴)(R⁵)—C(R⁴)(R⁵)—, or —C(R²)═C(R³)—C(R²)═C(R³)—;
m is 0, n is 1,

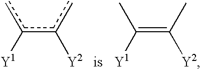

X¹ is —C(R²)═C(R³)—, —C(R⁴)(R⁵)—C(R⁴)(R⁵)—, —C(R²)═C(R³)—C(R⁴)(R⁵)—, —C(R⁴)(R⁵)—C(R²)═C(R³)—, —C(R⁴)(R⁵)—C(R⁴)(R⁵)—C(R²)═C(R³)—, C(R²)═C(R³)—C(R⁴)(R⁵)—C(R⁴)(R⁵)—, or —C(R²)═C(R³)—C(R²)═C(R³)—;
m is 1, n is 1,

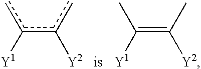

X¹ and X² are independently of each other —C(R⁴)(R⁵)—, —C(R⁴)(R⁵)—C(R⁴)(R⁵)—, or —C(R²)═C(R³)—;
m is 1, n is 1,

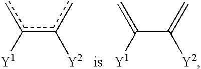

X¹ and X² are independently of each other —C(R²)—, —C(R³)—, —C(R³)—C(R⁴)(R⁵)—, or —C(R²)—C(R⁴)(R⁵)—;

### Y¹ is —OH, Y² is —CO—NR⁸R⁹, or Y¹¹; or

Y² is —OH, Y¹ is —CO—NR⁸R⁹, or Y¹¹; wherein
Y¹¹ is 2H-2- or 5-pyrrolyl, imidazolyl, 3- or 5-pyrazolyl, 2- or 4-thiazolyl, 2- or 4-oxazolyl, 3-isoxazolyl, 2- or 6-pyridyl, pyrazinyl, 3- or 6-pyridazinyl, triazinyl, 2-benzimidazolyl, 2-benzothiazolyl, 2-benzoxazolyl, 3- or 4-benzothiadiazolyl, 1-triazolyl, 3-indazolyl, 2-quinolyl, 1- or 3-isoquinolyl, 1- or 4-phthalazinyl, 2- or 3-quinoxalinyl, pteridinyl, each of which can optionally be substituted 1 to 7 times by halogen, C₁-C₁₈alkyl, C₃-C₈cycloalkyl, benzyl and/or phenoxycarbonyl; phenyl which can optionally be substituted by one or more —OR⁶, —SR⁷ and/or —NR⁸R⁹; C₂-C₁₂alkoxycarbonyl, optionally interrupted by one or more —O—, —S— and/or substituted by one or more hydroxyl groups; —OR⁶, —SR⁷, —SOR⁷, —SO₂R⁷ and/or —NR³R⁹; C₁-C₈alkanoyl, or benzoyl which can optionally be substituted by —OR⁶, —SR⁷, —SOR⁷, —SO₂R⁷, —NR⁸R⁹, morpholino and/or dimethylmorpholino; wherein R⁶, R⁷, R⁸ and/or R⁹ can optionally form 5-, 6- or 7-membered rings with further substituents on the heteroaromatic ring;

### X is —OH, or —NR⁸R⁹,

R¹ is —OH, C₃-C₈cycloalkoxy, C₁-C₁₈alkoxy, C₃-C₆alkenoxy, or C₁-C₈thioalkoxy which can optionally be substituted by one or more groups halogen, —OR⁶, —SR⁷ and/or —CN; C₆-C₂₄aryloxy, especially phenoxy, C₇-C₂₄aralkyloxy, especially benzyloxy, C₆-C₂₄thioaryloxy, especially thiophenoxy, or C₇-C₂₄thioaralkyloxy, especially thiobenzyloxy, which can optionally be substituted by one or more groups C₁-C₈alkyl, halogen, —CN, —OR⁶, —SR⁷ and/or —NR⁸R⁹;
R² and R³ are independently of each other hydrogen,

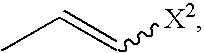

wherein X² is aryl, or heteroaryl, which can be substituted one to three times with C₁-C₈alkyl and/or C₁-C₈alkoxy; —OR⁶, —SR⁷ or —NR⁸R⁹; C₁-C₂₅alkyl which can optionally be substituted by one or more groups halogen, —OR⁶, phenyl, naphthyl and/or phenanthryl which can optionally be substituted by —OR⁶, —SR⁷ and/or —NR⁸R⁹; C₃-C₈cycloalkyl; C₂-C₂₀alkyl interrupted by one or more —O—, —S— and/or optionally substituted by one or more halogen, —OR⁶, phenyl which can optionally be substituted by —OR⁶, —SR⁷ and/or —NR⁸R⁹; aryl, or heteroaryl, such as Y¹¹, which can optionally be substituted by one or more groups C₁-C₈alkyl, phenyl, halogen, —OR⁶, —SR⁷ and/or —NR⁸R⁹; C₂-C₂₀alkanoyl, or benzoyl which can optionally be substituted by one or more groups C₁-C₈alkyl, phenyl, —OR⁶, —SR⁷ and/or —NR⁸R⁹; C₂-C₁₂alkoxycarbonyl optionally interrupted by one or more —O—, —S— and/or optionally substituted by one or more hydroxyl groups; phenoxycarbonyl which can optionally be substituted by C₁-C₈alkyl, halogen, phenyl, —OR⁶, —SR⁷ and/or —NR⁸R⁹; —CN, COOH, —CO—NR⁸R⁹, —NO₂, C₁-C₄haloalkyl, —S(O)₁₋₂—C₁-C₈alkyl or —S(O)₁₋₂-phenyl which can optionally be substituted by C₁-C₁₂alkyl; —SO₂O-phenyl which can optionally be substituted by C₁-C₁₂alkyl; wherein the substituents R¹, R², R³, Y and Y¹ can optionally form 5-, 6- or 7-membered rings with each other;
R⁴ and R⁵ are as defined for R², or R⁴ and R⁵ form 5-, 6- or 7-membered rings with each other, which may be substituted,
R⁶ is hydrogen, C₁-C₂₀alkyl, phenyl-C₁-C₃alkyl; C₁-C₈alkyl which is substituted by —OH, —SH, —CN, C₃-C₆alkenoxy, —OCH₂CH₂CN, —OCH₂CH₂(CO)O(C₁-C₄alkyl), —O(CO)—(C₁-C₄alkyl), —O(CO)-phenyl, —(CO)OH and/or —(CO)O(C₁-C₄alkyl); C₂-C₁₂alkyl which is interrupted by one or more —O—, —S—; —(CH₂CH₂O)ₙH, (CH₂CH₂O)ₙ(CO)—(C₁-C₈alkyl), C₁-C₈alkanoyl, C₂-C₁₂alkenyl, C₃-C₆alkenoyl, C₃-C₈cycloalkyl; benzoyl which can optionally be substituted by one or more groups C₁-C₈alkyl, halogen, —OH and/or C₁-C₄alkoxy; phenyl or naphthyl each of which can optionally be substituted by halogen, —OH, C₁-C₁₂alkyl, C₁-C₁₂-alkoxy, phenoxy, C₁-C₁₂alkylsulfanilyl, phenylsulfanilyl, —N(C₁-C₁₂alkyl)₂ and/or diphenylamino;
R⁷ is hydrogen, C₁-C₂₀alkyl, C₂-C₁₂alkenyl, C₃-C₈cycloalkyl, phenyl-C₁-C₃alkyl; C₁-C₆alkyl which is substituted by —OH, —SH, —CN, C₃-C₆alkenoxy, —OCH₂CH₂CN, —OCH₂CH₂(CO)O(C₁-C₄alkyl), —O(CO)—(C₁-C₄alkyl), —O(CO)-phenyl, —(CO)OH or —(CO)O(C₁-C₄alkyl); C₂-C₁₂alkyl which is interrupted by one or more —O—, or —S—; —(CH₂CH₂O)ₜH, —(CH₂CH₂O)ₜ(CO)—(C₁-C₈alkyl), C₁-C₈alkanoyl, C₂-C₁₂alkenyl, C₃-C₆alkenoyl; benzoyl which can optionally be substituted by one or more groups C₁-C₈alkyl, halogen, —OH, C₁-C₄alkoxy, or C₁-C₄alkylsulfanilyl; phenyl or naphthyl, each of which can optionally be substituted by halogen, C₁-C₁₂alkyl, C₁-C₁₂alkoxy, phenyl-C₁-C₃alkyloxy, phenoxy, C₁-C₁₂alkylsulfanilyl, phenylsulfanilyl, —N(C₁-C₁₂alkyl)₂, diphenylamino, —(CO)O(C₁-C₈alkyl), —(CO)—C₁-C₈alkyl, or —(CO)N(C₁-C₈alkyl)₂;
t is 1 to 20;
R⁸ and R⁹ are independently of each other hydrogen, C₁-C₂₀alkyl, C₂-C₄-hydroxyalkyl, C₂-C₁₀-alkoxyalkyl, C₂-C₅alkenyl, C₃-C₈cycloalkyl, C₇-C₂₄aralkyl, especially phenyl-C₁-C₃alkyl, C₁-C₈alkanoyl, C₃-C₁₂-alkenoyl, formyl, benzoyl; C₆-C₂₄aryl, especially phenyl, or naphthyl, each of which can optionally be substituted by C₁-C₁₂alkyl, benzoyl, or C₁-C₁₂alkoxy; or R⁸ and R⁹ together are C₂-C₈alkylene optionally interrupted by —O—, —S— or —NR¹⁶³— and/or optionally substituted by hydroxyl, C₁-C₄alkoxy, C₂-C₄alkanoyloxy, benzoyloxy, C₁-C₁₂alkylsulfanilyl, or phenylsulfanilyl which can optionally be substituted by one or more C₁-C₈alkyl, halogen, —OH and/or C₁-C₄alkoxy; or R³ and/or R⁹ together with a substituent, which is adjacent to the group —CO—NR⁸R⁹, or —NR³R⁹, form 5-, 6- or 7-membered rings, which may be substituted; R¹⁶³ is a hydrogen atom, a C₁-C₂₅alkyl group, which might be interrupted by —O—, a cycloalkyl group, an aralkyl group, an aryl group, or a heterocyclic group.
with the proviso that the following compounds are excluded:

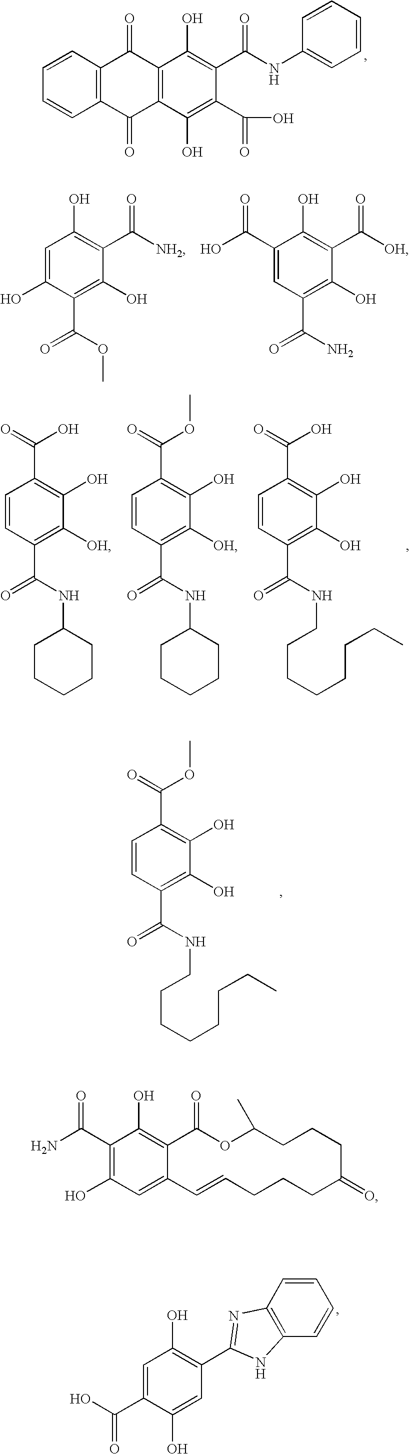

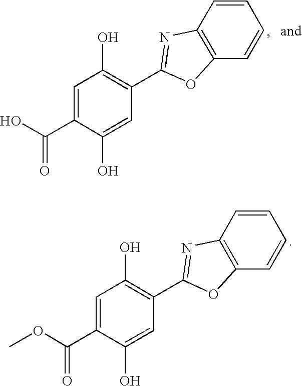

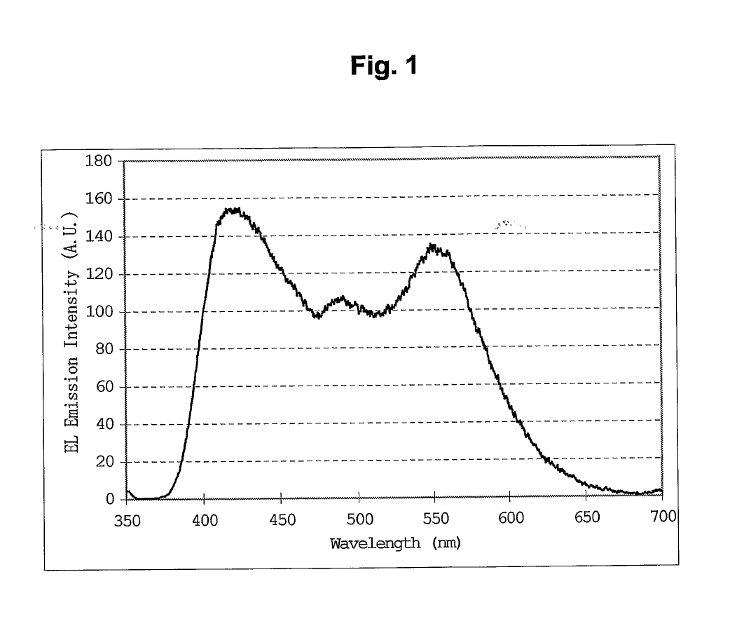

FIG. 1 is an EL emission spectrum of an EL device comprising 4,4′-N,N′-dicarbazole-biphenyl (CBP) and 4,7-dihydroxy-1-oxo-3-phenyl-2-propyl-2,3-dihydro-1H-isoindole-5-carboxylic acid isopropyl ester (compound of example 1) as light emitting layer (example 14).

If the compound of formula I is a compound of formula

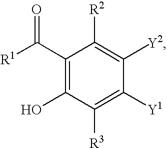

those compounds are preferred, wherein Y² is —OH, wherein those compounds are especially preferred, wherein Y² is —OH and Y¹ is —CONR⁸R⁹.

If in the above formula Y² is —OH, those compounds are preferred, wherein R² and/or R³ are different from hydrogen.

If in the above formula R¹ is —OH and Y² is —OH, and Y is benzimidazolyl, benzthiazolyl, or benzoxazolyl, R² and/or R³ are different from hydrogen.

If the compound of formula I is a compound of formula

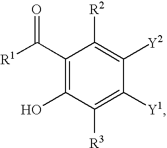

wherein Y¹ is —OH, those compounds are preferred, wherein R³ is different from hydrogen, —CONH₂, and —COOH.

If the compound of formula I is a compound of formula

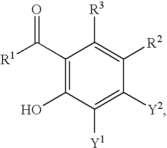

wherein Y¹ is —OH, those compounds are preferred, wherein R² and/or R³ are different from hydrogen, and/or Y² is different from —CONR⁸R⁹.

In a preferred embodiment the present invention is directed to six-membered ring compounds of the formula

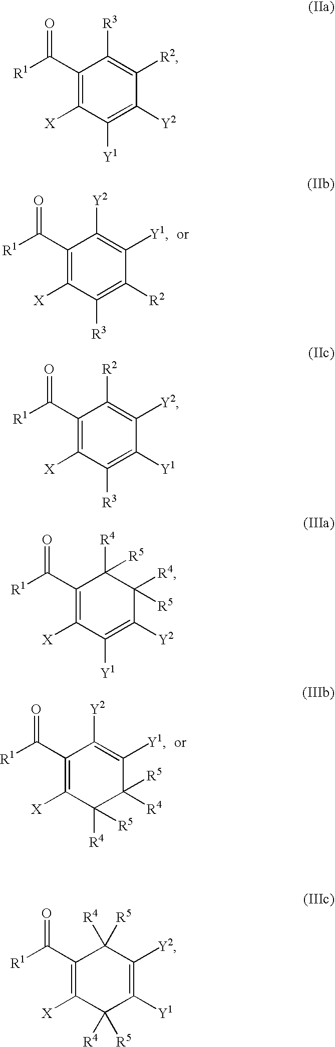

to seven-membered ring compounds of the formula

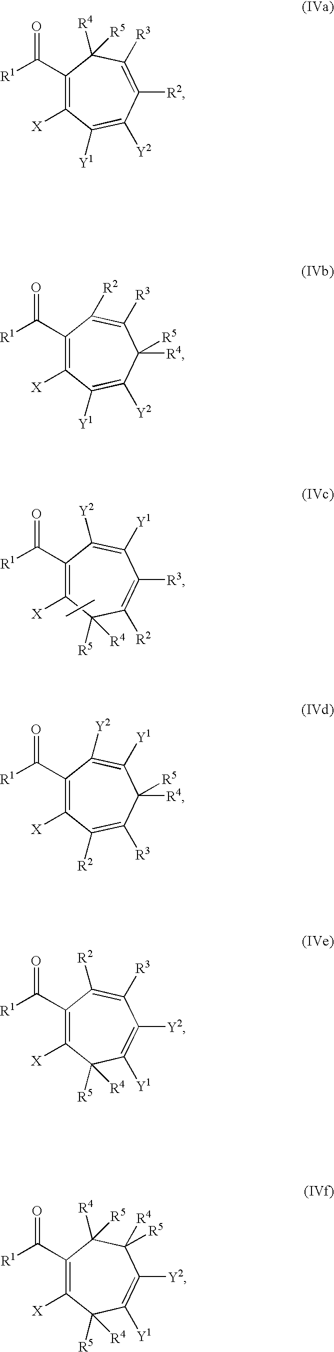

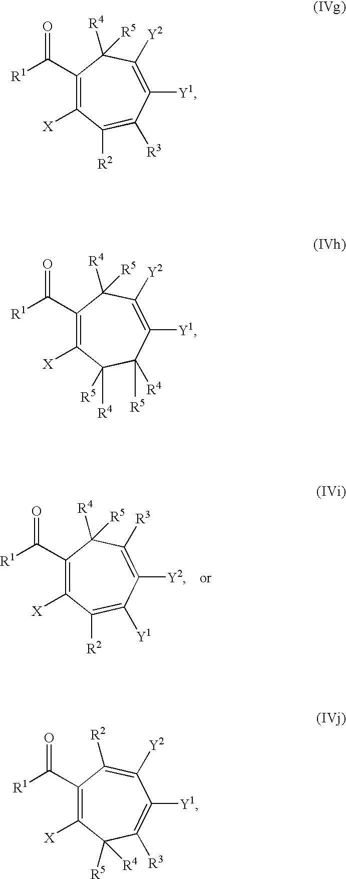

and to eight-membered ring compounds of the formula

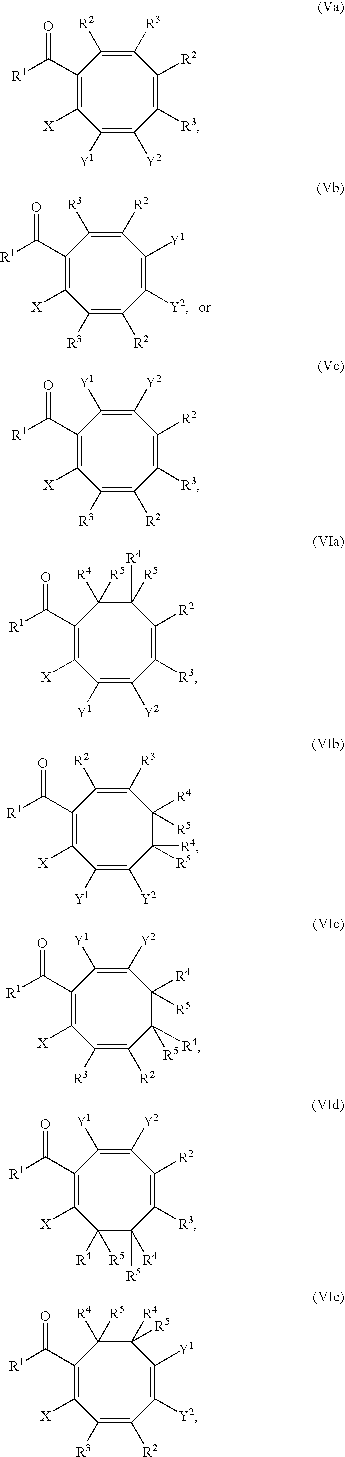

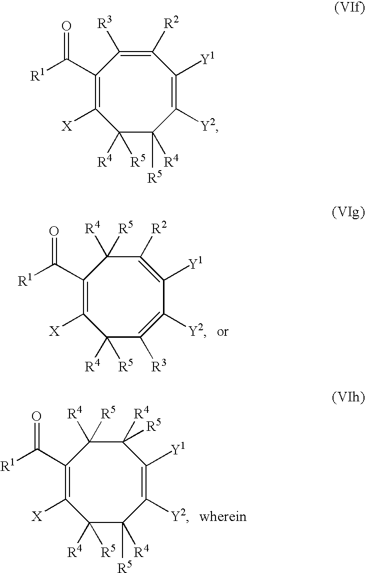

### Y¹ is —OH, Y² is —CO—NR⁸R⁹, or Y¹¹; or

Y² is —OH, Y¹ is —CO—NR⁸R⁹, or Y¹¹; wherein
Y¹¹ is 2H-2- or 5-pyrrolyl, imidazolyl, 3- or 5-pyrazolyl, 2- or 4-thiazolyl, 2- or 4-oxazolyl, 3-isoxazolyl, 2- or 6-pyridyl, pyrazinyl, 3- or 6-pyridazinyl, triazinyl, 2-benzimidazolyl, 2-benzothiazolyl, 2-benzoxazolyl, 3- or 4-benzothiadiazolyl, 1-triazolyl, each of which can optionally be substituted 1 to 4 times by halogen, C₁-C₁₈alkyl, benzyl and/or phenoxycarbonyl; phenyl which can optionally be substituted by one or more —OR⁶, —SR⁷ and/or —NR⁸R⁹; C₂-C₁₂alkoxycarbonyl, optionally interrupted by one or more —O—, —S— and/or substituted by one or more hydroxyl groups; —OR⁶ and/or —NR⁸R⁹; wherein R⁶, R⁷, R⁸ and/or R⁹ can optionally form 5-, 6- or 7-membered rings with further substituents on the heteroaromatic ring;

### X is —OH, or —NR³R⁹,

R¹ is —OH, C₃-C₈cycloalkoxy, C₁-C₁₈alkoxy, C₃-C₆alkenoxy, or C₁-C₈thioalkoxy which can optionally be substituted by one or more groups halogen, —OR⁶, —SR⁷ and/or —CN; C₆-C₂₄aryloxy, especially phenoxy, C₇-C₂₄aralkyloxy, especially benzyloxy, C₆-C₂₄thioaryloxy, especially thiophenoxy, or C₇-C₂₄thioaralkyloxy, especially thiobenzyloxy, which can optionally be substituted by one or more groups C₁-C₈alkyl, halogen, —CN, —OR⁶, —SR⁷ and/or —NR⁸R⁹;
R² and R³ are independently of each other hydrogen,

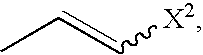

wherein X² is aryl, or heteroaryl, which can be substituted one to three times with C₁-C₈alkyl and/or C₁-C₈alkoxy; —OR⁶, —SR⁷ or —NR³R⁹; C₁-C₁₈alkyl which can optionally be substituted by one or more groups halogen, —OR⁶, phenyl, naphthyl and/or phenanthryl which can optionally be substituted by —OR⁶, —SR⁷ and/or —NR⁸R⁹; C₃-C₈cycloalkyl; C₂-C₁₂alkyl interrupted by one or more —O—, —S— and/or optionally substituted by one or more halogen, —OR⁶, phenyl which can optionally be substituted by —OR⁶, —SR⁷ and/or —NR⁸R⁹; aryl or heteroaryl, especially phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, phenyl, halogen, —OR⁶, —SR⁷ and/or —NR⁸R⁹; C₂-C₁₂alkanoyl; C₂-C₁₂alkoxycarbonyl optionally interrupted by one or more —O—, —S— and/or optionally substituted by one or more hydroxyl groups; phenoxycarbonyl which can optionally be substituted by C₁-C₈alkyl, halogen, phenyl, —OR⁶, —SR⁷ and/or —NR⁸R⁹, —CO—NR⁶R⁹, C₁-C₄haloalkyl; wherein the substituents R¹ and R², or R³; R² and R³; R⁴ and R⁵; R¹, R², R³, R⁴, or R⁵ and X, Y¹, or Y² can optionally form 5-, 6- or 7-membered rings with each other, which can optionally be substituted;
R⁴ and R⁵ are as defined for R²,
R⁶ is hydrogen, C₁-C₁₂alkyl, phenyl-C₁-C₃alkyl; C₁-C₈alkyl which is substituted by —OH, —SH, —CN; C₂-C₁₂alkyl which is interrupted by one or more —O—, —S—; C₁-C₈alkanoyl; phenyl or naphthyl each of which can optionally be substituted by halogen, —OH, C₁-C₁₂alkyl, C₁-C₁₂-alkoxy, phenoxy, C₁-C₁₂alkylsulfanilyl, phenylsulfanilyl, —N(C₁-C₁₂alkyl)₂ and/or diphenylamino;
R⁷ is hydrogen, C₁-C₁₂alkyl; C₁-C₈alkyl which is substituted by —OH, —SH, —CN; C₂-C₁₂alkyl which is interrupted by one or more —O—, or —S—; phenyl or naphthyl, each of which can optionally be substituted by halogen, C₁-C₁₂alkyl, C₁-C₁₂alkoxy, phenyl-C₁-C₃alkyloxy, phenoxy, C₁-C₁₂alkylsulfanilyl, phenylsulfanilyl, —N(C₁-C₁₂alkyl)₂, diphenylamino;
R⁸ and R⁹ are independently of each other hydrogen, C₁-C₂₅alkyl, C₂-C₄-hydroxyalkyl, C₂-C₁₀-alkoxyalkyl, C₁-C₈alkanoyl, formyl, benzoyl; C₇-C₂₄aralkyl, especially phenyl-C₁-C₃alkyl, C₆-C₂₄aryl, especially phenyl, or naphthyl, each of which can optionally be substituted by C₁-C₁₂alkyl, benzoyl, or C₁-C₁₂alkoxy; or R⁸ and R⁹ together are C₂-C₈alkylene optionally interrupted by —O—, —S— or —NR¹⁶³— and/or optionally substituted by hydroxyl, C₁-C₄alkoxy; or
R⁸ and/or R⁹ together with a substituent, which is adjacent to the group —CO—NR⁸R⁹, or —NR⁸R⁹, form 5-, 6- or 7-membered rings, which may be substituted; R¹⁶³ is a hydrogen atom, a C₁-C₂₅alkyl group, which might be interrupted by —O—, a cycloalkyl group, an aralkyl group, an aryl group, or a heterocyclic group.

The following compounds of formula

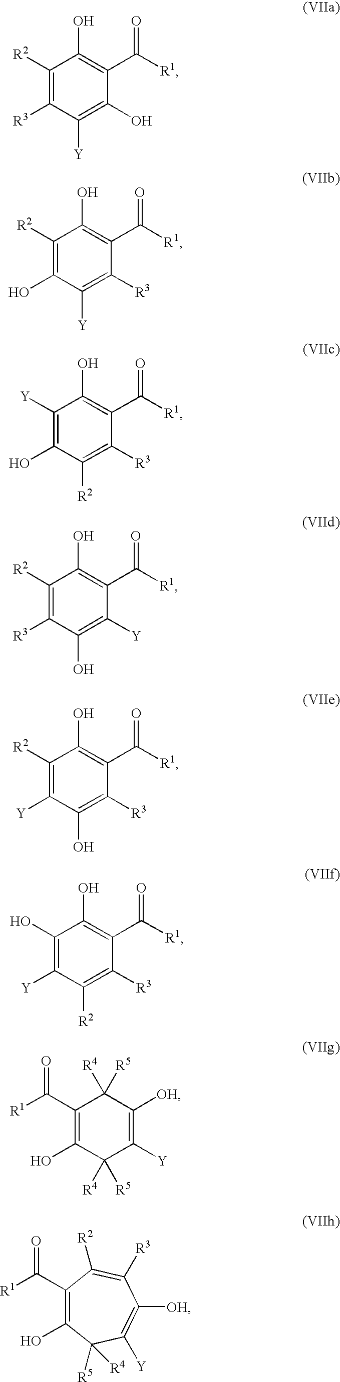

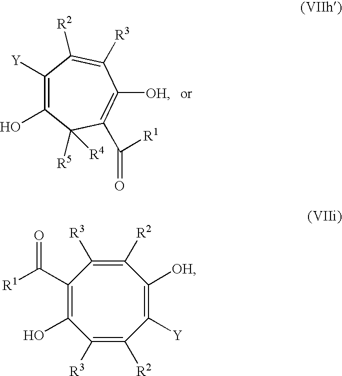

are especially preferred, wherein
Y is —CO—NR⁸R⁹ imidazolyl, oxazolyl, thiazolyl, benzimidazolyl, benzoxazolyl or benzothiazolyl;
R¹ is —OH, C₆-C₂₄aryloxy, especially phenoxy, which can be substituted one to three times with C₁-C₈-alkyl, or C₁-C₈alkoxy; C₇-C₃₀aralkyloxy, especially benzyloxy, which can be substituted one to three times with C₁-C₈-alkyl, or C₁-C₈alkoxy; C₁-C₁₈alkoxy which optionally forms 5- or 6-membered rings with adjacent substituents;
R² and R³ are independently of each other R² and R³ are independently of each other hydrogen, C₁-C₁₈alkoxy, C₁-C₁₈alkyl,

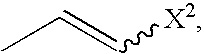

wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, C₆-C₂₄aryloxy, especially phenoxy, which can be substituted one to three times with C₁-C₈alkyl, C₇-C₂₄aralkyloxy, especially benzyloxy, which can be substituted one to three times with C₁-C₈alkyl, C₇-C₂₄aralkyl, especially benzyl, naphthylmethyl, or phenanthrylmethyl, which can be substituted one to three times with C₁-C₈alkyl; wherein R² and R³ optionally form 5-, 6- or 7-membered rings with substituents R², R³ or Y;
R⁴ and R⁵ are independently of each other hydrogen, C₁-C₂₅alkyl, or R⁴ and R⁵ together form a C₅-C₇cycloalkyl ring, especially cyclohexyl ring, which can be substituted one to three times with C₁-C₈alkyl;
R³ and R⁹ are independently of each other hydrogen, C₁-C₂₅alkyl, or C₆-C₂₄aryl, especially phenyl, which can be substituted one to three times with C₁-C₈alkyl, C₇-C₂₄aralkyl, especially benzyl, which can be substituted one to three times with C₁-C₈alkyl; or R³ and R⁹ together are C₂-C₈alkylene optionally substituted by C₁-C₈alkyl and/or optionally interrupted by —O—, —S—or —NR¹⁶³—, especially

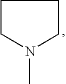

or morpholino; or if Y is —CO—NR⁸R⁹, R⁸ and R² or R³ form a five membered ring

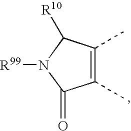

wherein R⁹⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³, wherein
R³³ and R³⁴ independently from each other stand for hydrogen or C₁-C₄alkyl, or phenyl, which can be substituted one to three times with C₁-C₄alkyl,
A³ stands for aryl or heteroaryl, in particular phenyl or 1- or 2-naphthyl, which can be substituted one to three times with C₁-C₈alkyl and/or C₁-C₈alkoxy, and s stands for 0, 1, 2, 3 or 4,
R⁹ is as defined above and R¹⁰ is C₁-C₁₈alkyl; especially aryl, or heteroaryl, especially phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, halogen, or —OR⁶;
R⁶ is C₁-C₈alkyl; and
R¹⁶³ is a hydrogen atom, a C₁-C₂₅alkyl group, which might be interrupted by —O—, a cycloalkyl group, an aralkyl group, an aryl group, or a heterocyclic group.

If Y is heteroaryl, it is preferably 2H-2- or 5-pyrrolyl, imidazolyl, 3- or 5-pyrazolyl, 2- or 4-thiazolyl, 2- or 4-oxazolyl, 3-isoxazolyl, 2- or 6-pyridyl, pyrazinyl, 3- or 6-pyridazinyl, triazinyl, 2-benzimidazolyl, 2-benzothiazolyl, 2-benzoxazolyl, 3- or 4-benzothiadiazolyl, 1-triazolyl, each of which can optionally be substituted 1 to 4 times by halogen, or C₁-C₁₈alkyl. If Y is —CO—NR⁸R⁹, groups —CO—NR⁸R⁹ are preferred, wherein R⁸ and R⁹ are independently of each other hydrogen, C₁-C₂₅alkyl, or C₆-C₂₄aryl, especially phenyl, which can be substituted one to three times with C₁-C₈alkyl, C₇-C₂₄aralkyl, especially benzyl, which can be substituted one to three times with C₁-C₈alkyl; or R⁸ and R⁹ together are C₂-C₈alkylene optionally substituted by C₁-C₈alkyl and/or optionally interrupted by —O—, —S— or —NR¹⁶³—, especially

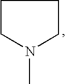

or morpholino; or R⁸ and R² or R³ form a five membered ring

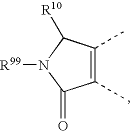

wherein R⁹⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³, wherein
R³³ and R³⁴ independently from each other stand for hydrogen or C₁-C₄alkyl, or phenyl, which can be substituted one to three times with C₁-C₄alkyl,
A³ stands for aryl or heteroaryl, in particular phenyl or 1- or 2-naphthyl, which can be substituted one to three times with C₁-C₈alkyl and/or C₁-C₈alkoxy, and s stands for 0, 1, 2, 3 or 4,
and R¹⁰ is C₁-C₁₈alkyl; especially aryl, or heteroaryl, especially phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, halogen, or —OR⁶;
R⁶ is C₁-C₈alkyl; and
R¹⁶³ is a hydrogen atom, a C₁-C₂₅alkyl group, which might be interrupted by —O—, a cycloalkyl group, an aralkyl group, an aryl group, or a heterocyclic group.

R¹ is preferably —OH, C₇-C₃₀aralkyloxy, especially benzyloxy, which can be substituted one to three times with C₁-C₈-alkyl, or C₁-C₈-alkoxy, C₆-C₂₄aryloxy, especially phenoxy, which can be substituted one to three times with C₁-C₈-alkyl, or C₁-C₈alkoxy.

R² and R³ are preferably independently of each other hydrogen, C₁-C₁₈alkoxy, C₁-C₁₈alkyl,

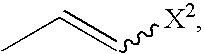

wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, C₆-C₂₄aryloxy, especially phenoxy, which can be substituted one to three times with C₁-C₈alkyl, C₇-C₂₄aralkyloxy, especially benzyloxy, which can be substituted one to three times with C₁-C₈alkyl, C₇-C₂₄aralkyl, especially benzyl, naphthylmethyl, or phenanthrylmethyl, which can be substituted one to three times with C₁-C₈alkyl; wherein R² and R³ optionally form 5-, 6- or 7-membered rings with substituents R², R³ or Y;
R⁴ and R⁵ are preferably independently of each other hydrogen, C₁-C₂₅alkyl, or R⁴ and R⁵ together form a C₅-C₇cycloalkyl ring, especially cyclohexyl ring, which can be substituted one to three times with C₁-C₈alkyl,
If R², R³ and R¹⁰ stand for “aryl”, the “aryl group” is typically C₆-C₂₄aryl, such as phenyl, indenyl, azulenyl, naphthyl, biphenyl, as-indacenyl, s-indacenyl, acenaphthylenyl, fluorenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Preferred examples of C₆-C₁₄aryl are phenyl, 1-naphthyl, 2-naphthyl, 3- or 4-biphenyl, 2- or 9-fluorenyl or 9-phenanthryl, which may be unsubstituted or substituted.

If R², R³ and R¹⁰ stand for “aryl”, they are especially a group of formula

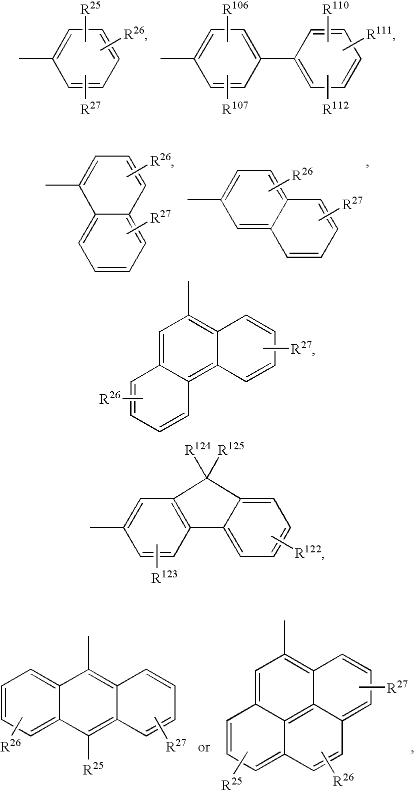

wherein
R²⁵, R²⁶, R²⁷, R¹⁰⁶, R¹⁰⁷, R¹¹⁰, R¹¹¹, R¹¹², R¹²², R¹²³, R¹²⁴ and R¹²⁵ are as defined below, especially a group of formula

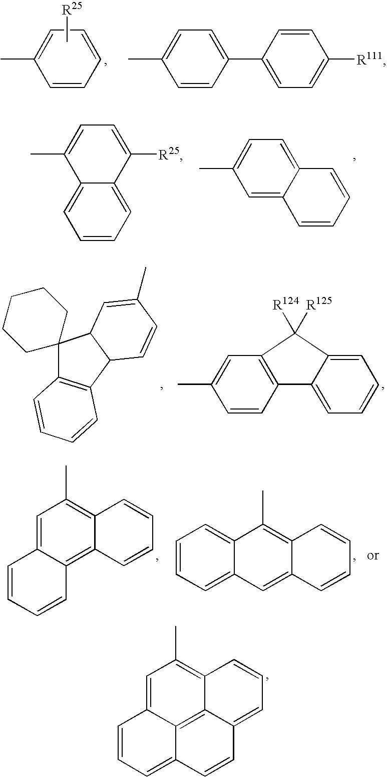

wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl.

If R², R³ and R¹⁰ stand for “heteroaryl”, the term “heteroaryl” means a ring, wherein nitrogen, oxygen or sulfur are the possible hetero atoms, and is typically an unsaturated heterocyclic radical with five to 24 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono-, bi, or tricyclic heterocyclic radicals, which may be unsubstituted or substituted, especially by one to three C₁-C₈alkyl groups.

If R², R³ and R¹⁰ stand for “heteroaryl”, they are especially a group of formula

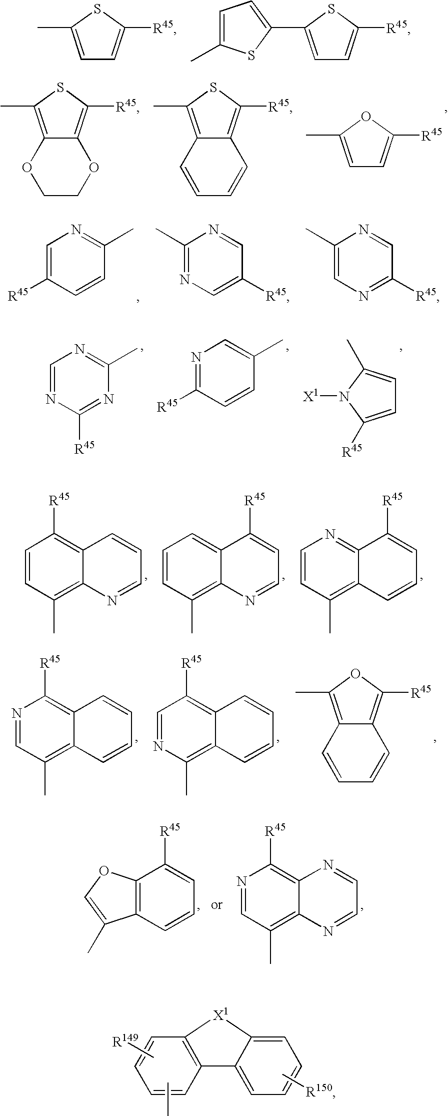

especially

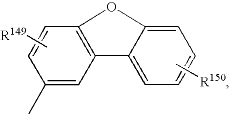

or

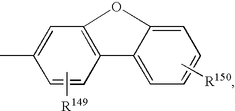

wherein X¹, R⁴⁵, R¹⁴⁹ and R¹⁵⁰ are as defined below, in particular

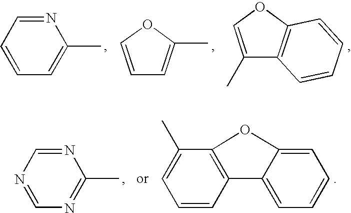

In another embodiment of the present invention compounds of formula

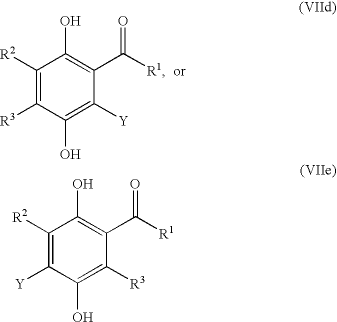

are preferred, wherein
R¹ is —OH, C₇-C₃₀aralkyloxy, especially benzyloxy, which can be substituted one to three times with C₁-C₈-alkyl, CO—C₂₋₄aryloxy, especially phenoxy, which can be substituted one to three times with C₁-C₈-alkyl, or C₁-C₁₈alkoxy,

R² and R³ are independently of each other H, C₁-C₁₈alkyl,

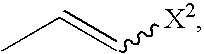

wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy,
Y stands for —CO—NR⁸R⁹, wherein
R⁸ and R⁹ are independently of each other hydrogen, C₁-C₂₅alkyl, or C₆-C₂₄aryl, especially phenyl, which can be substituted one to three times with C₁-C₈-alkyl, C₇-C₂₄aralkyl, especially benzyl, which can be substituted one to three times with C₁-C₈-alkyl; or R⁸ and R⁹ together are C₂-C₈alkylene optionally substituted by C₁-C₈alkyl and/or optionally interrupted by —O—, —S—or —NR¹⁶³—, especially

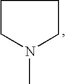

or morpholino; or

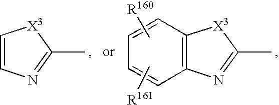

wherein X³ is O, S, or NR¹⁶³, wherein R¹⁶³ is a hydrogen atom, a C₁-C₂₅alkyl group, which might be interrupted by —O—, a cycloalkyl group, an aralkyl group, an aryl group, or a heterocyclic group, and
R¹⁶⁰ and R¹⁶¹ may be the same or different and are selected from hydrogen, C₁-C₂₅alkyl, cycloalkyl, aralkyl, alkenyl, cycloalkenyl, alkynyl, hydroxyl, a mercapto group, alkoxy, alkylthio, an aryl ether group, an aryl thioether group, aryl, a heterocyclic group, halogen, haloalkyl, haloalkenyl, haloalkynyl, a cyano group, an aldehyde group, a carbonyl group, a carboxyl group, an ester group, a carbamoyl group, a group NR¹²⁷R¹²⁸, wherein R¹²⁷ and R¹²⁸ independently of each other stand for a hydrogen atom, an alkyl group, an optionally substituted cycloalkyl group, an optionally substituted aryl group, an optionally substituted heteroaryl group, an optionally substituted heterocyclic group, an aralkyl group, or R¹²⁷ and R¹²⁸ together with the nitrogen atom to which they are bonded form a five or six membered heterocyclic ring, which can be condensed by one or two optionally substituted phenyl groups, a nitro group, a silyl group, a siloxanyl group, a substituted or unsubstituted vinyl group; or
R³ forms 5-, 6- or 7-membered rings with R¹, which can optionally be substituted by aryl, or heteroaryl groups, especially phenyl, biphenyl, naphthyl, fluorenyl, anthryl, pyrenyl, phenanthryl, pyridyl, furanyl, benzofuranyl, triazinyl, or dibenzofuranyl, which can be substituted one to three times with C₁-C₈-alkyl; or which can be condensed one time by phenyl which can be substituted one to three times with C₁-C₄-alkyl.

Preferably at least one of R² and R³, more preferred both R² and R³ are

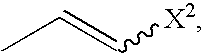

wherein X² is aryl, or heteroaryl, which can be substituted one to three times with C₁-C₈alkyl and/or C₁-C₈alkoxy; aryl or heteroaryl.

In said embodiment compounds of formula VIId and VIIe are more preferred, wherein R¹ is benzyloxy, which can be substituted one to three times with C₁-C₈-alkyl, phenoxy, which can be substituted one to three times with C₁-C₈-alkyl, or C₁-C₁₈alkoxy,

R² and R³ are independently of each other

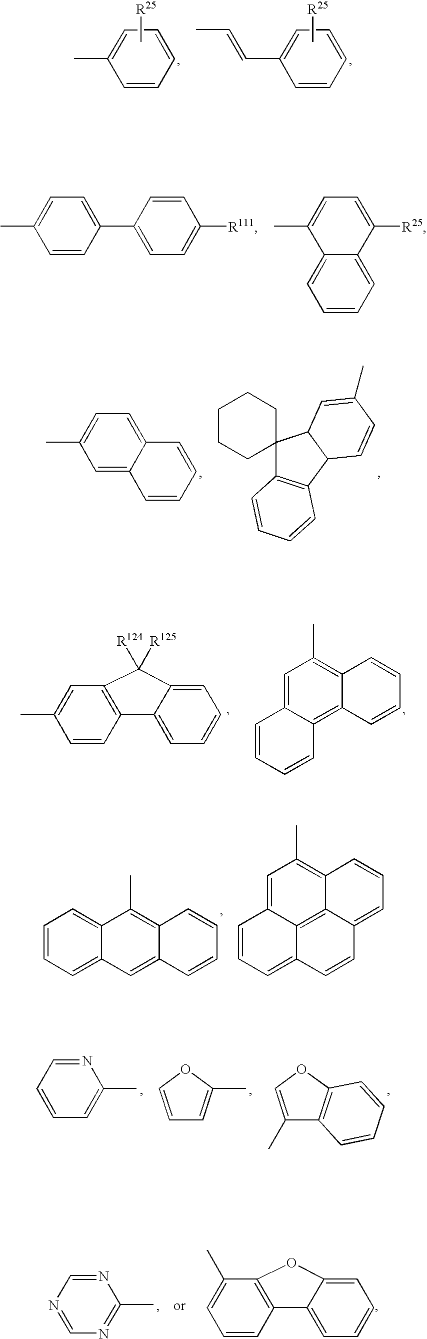

wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl;
Y stand for —CO—NR⁸R⁹, wherein
R⁸ and R⁹ are independently of each other hydrogen, C₁-C₂₅alkyl, phenyl, which can be substituted one to three times with C₁-C₈-alkyl, or

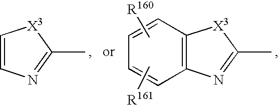

wherein X³ is O, or NR¹⁶³, wherein R¹⁶³ is a C₁-C₂₅alkyl group, which might be interrupted by —O—, a cycloalkyl group, or a phenyl group, and
R¹⁶⁰ and R¹⁶¹ may be the same or different and are selected from hydrogen, or C₁-C₂₅alkyl; or
R⁸ and R⁹ together are C₂-C₈alkylene optionally substituted by C₁-C₈alkyl and/or optionally interrupted by —O—, —S— or —NR¹⁶³—, especially

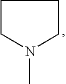

or morpholino; or
R³ and R¹ together form a ring and are a group —CHR¹⁰⁰—O—, wherein R¹⁰⁰ is hydrogen, C₁-C₁₈alkyl, C₆-C₂₄aryl, especially phenyl, biphenyl, naphthyl, fluorenyl, or phenanthryl, which can be substituted one to three times with C₁-C₈-alkyl, or
R³ and R¹ together form a ring and are a group —CR¹⁰³═CR¹⁰⁴—O—, wherein R¹⁰³ and R¹⁰⁴ are independently of each other hydrogen, C₁-C₁₈alkyl, C₆-C₂₄aryl, especially phenyl, biphenyl, naphthyl, fluorenyl, or phenanthryl, which can be substituted one to three times with C₁-C₈-alkyl, or
R¹⁰³ and R¹⁰⁴ together form a ring and are a group

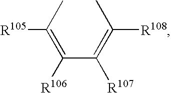

wherein R¹⁰⁵, R¹⁰⁶, R¹⁰⁷ and R¹⁰⁸ are independently of each other hydrogen, or C₁-C₁₈alkyl.

The following compounds of formula (VIIe) and (VIId) are most preferred:

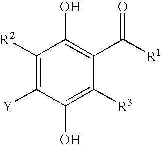

| Compound | R¹ | R² | R³ | Y |
|---|---|---|---|---|
| C-1 | —O—C₂H₅ | 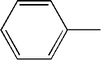 | 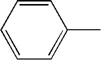 | —C(═O)NH(n-C₆H₁₃) |
| C-2 | —OtBu | 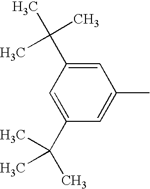 | 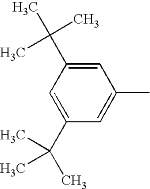 | —C(═O)N(CH₃)₂ |
| C-3 | —OtBu | 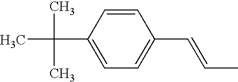 | 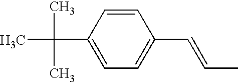 | —C(═O)NHCH₃ |
| C-4 | —OtBu | 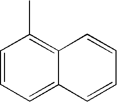 | 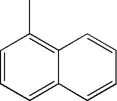 | —C(═O)N(CH₃)₂ |
| C-5 | —O—C₂H₅ | 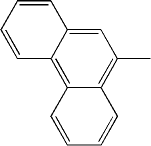 | 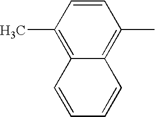 | —C(═O)NH(n-C₆H₁₃) |
| C-6 | —OtBu | 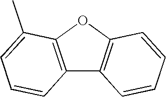 | 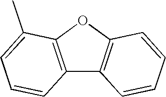 | —C(═O)N(CH₃)₂ |
| C-7 | —OtBu | 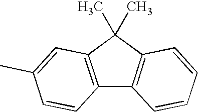 | 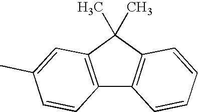 | —C(═O)N(CH₃)₂ |
| C-8 | —O-i-C₃H₇ | 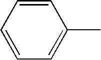 | 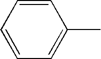 | 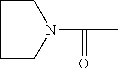 |
| C-9 | —O-i-C₃H₇ | 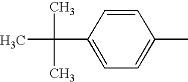 | 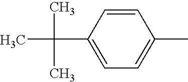 | 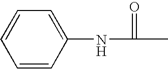 |
| C-10 | ¹⁾ | 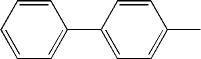 | ¹⁾ | —C(═O)N(CH₃)₂ |
| C-11 | ²⁾ | 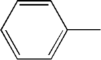 | ²⁾ | —C(═O)NH(n-C₆H₁₃) |
| C-12 | ³⁾ | 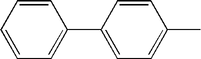 | ³⁾ | 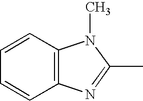 |
| C-13 | ⁴⁾ | 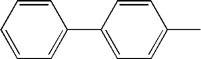 | ⁴⁾ | —C(═O)N(CH₃)₂ |
| C-14 | —O—C₂H₅ | 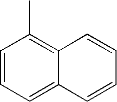 | 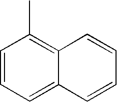 | 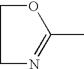 |
| C-15 | —OC₂H₅ | H | —C(O)NH-n-hexyl | H |
| C-16 | —OC₂H₅ | H | —C(O)morpholino | H |
| C-17 | —OCH₃ | —OPh | —C(O)NH-n-hexyl | —OPh |
| C-18 | —OC₂H₅ | H | —C(O)NHCH₂Ph | H |

¹⁾R³ and R¹ together form a ring and are a group —CH₂—O—.

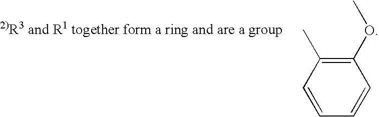

³⁾R³ and R¹ together form a ring and are a group —CH═CH—O—.
⁴⁾R¹¹ and R¹ together form a ring and are a group —CH₂CH₂—O—.

| | |
|---|---|
| | (VIId) |
| 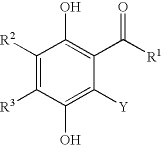 | |

| Compound | R¹ | R² | Y | R³ |
|---|---|---|---|---|
| C-15 | —O—C₂H₅ | 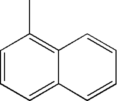 | —C(═O)NHC₂H₅ | 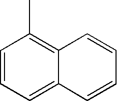 |

In another preferred embodiment the present invention relates to compounds of formula

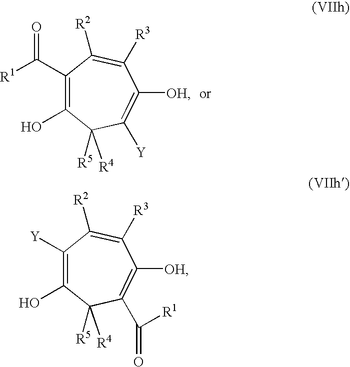

wherein Y and R¹ are as defined above,
R² and R³ are independently of each other H, C₁-C₁₈alkyl,

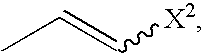

wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, or
R² and R³ together form a ring and are a group

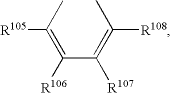

wherein R¹⁰⁵, R¹⁰⁶, R¹⁰⁷ and R¹⁰⁸ are independently of each other hydrogen, or C₁-C₁₈alkyl;
R⁴ and R⁵ are independently of each other hydrogen, C₁-C₂₅alkyl, which might be interrupted by —O—, or R⁴ and R⁵ together form a cyclohexane ring, which can optionally be substituted by C₁-C₈alkyl.

In said embodiment compounds of formula VIIh, or VIIh′ are more preferred, wherein R¹ is benzyloxy, which can be substituted one to three times with C₁-C₈-alkyl, phenoxy, which can be substituted one to three times with C₁-C₈-alkyl, or C₁-C₁₈alkoxy;

R² and R³ are independently of each other H,

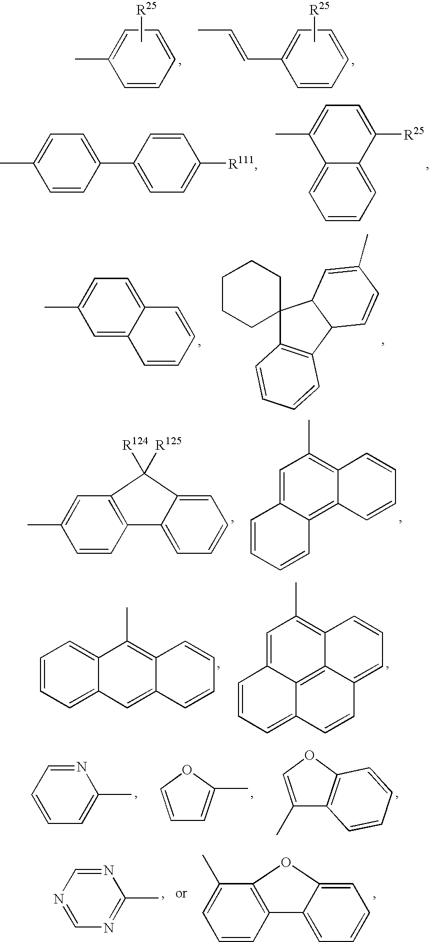

wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl;
Y stand for —CO—NR⁸R⁹, wherein
R⁸ and R⁹ are independently of each other hydrogen, C₁-C₂₅alkyl, phenyl, which can be substituted one to three times with C₁-C₈-alkyl, or

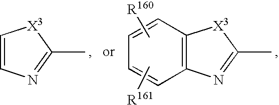

wherein X³ is O, or NR¹⁶³, wherein R¹⁶³ is a C₁-C₂₅alkyl group, which might be interrupted by —O—, a cycloalkyl group, or a phenyl group, and
R¹⁶⁰ and R¹⁶¹ may be the same or different and are selected from hydrogen, or C₁-C₂₅alkyl; or
R⁸ and R⁹ together are C₂-C₈alkylene optionally substituted by C₁-C₈alkyl and/or optionally interrupted by —O—, —S— or —NR¹⁶³—, especially

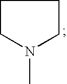

with the proviso that at least one of R² and R³ is different from hydrogen.

Preferably both, R² and R³, are different from hydrogen.

The following compound of formula VIIh′ is most preferred:

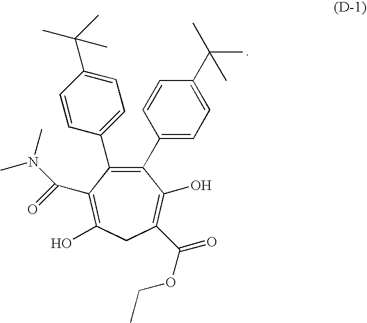

In another preferred embodiment the present invention relates to compounds of formula

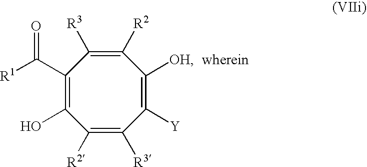

Y and R¹ are as defined above,
R², R³, R² and R³ are independently of each other hydrogen, C₁-C₁₈alkyl,

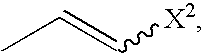

wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, or
R² and R³ together form a ring and are a group

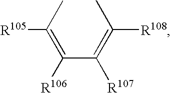

wherein R¹⁰⁵, R¹⁰⁶, R¹⁰⁷ and R¹⁰⁸ are independently of each other hydrogen, or C₁-C₁₈alkyl;
R⁴ and R⁵ are independently of each other hydrogen, C₁-C₂₅alkyl, which might be interrupted by —O—, or R⁴ and R⁵ together form a cyclohexane ring, which can optionally be substituted by C₁-C₈alkyl, with the proviso that at least one, preferably two of R², R³, R^{2′} and R^{3′} are different from hydrogen.

In said embodiment compounds of formula VIIi are more preferred, wherein R² and R^{2′} are hydrogen and R³ and R^{3′} are different from hydrogen; or wherein R³ and R^{3′} are hydrogen and R² and R^{2′} are different from hydrogen.

In said embodiment compounds of formula VIIi are more preferred, wherein

R¹ is benzyloxy, which can be substituted one to three times with C₁-C₈-alkyl, phenoxy, which can be substituted one to three times with C₁-C₈-alkyl, or C₁-C₁₈alkoxy; R², R³, R^{2′} and R^{3′} are independently of each other H,

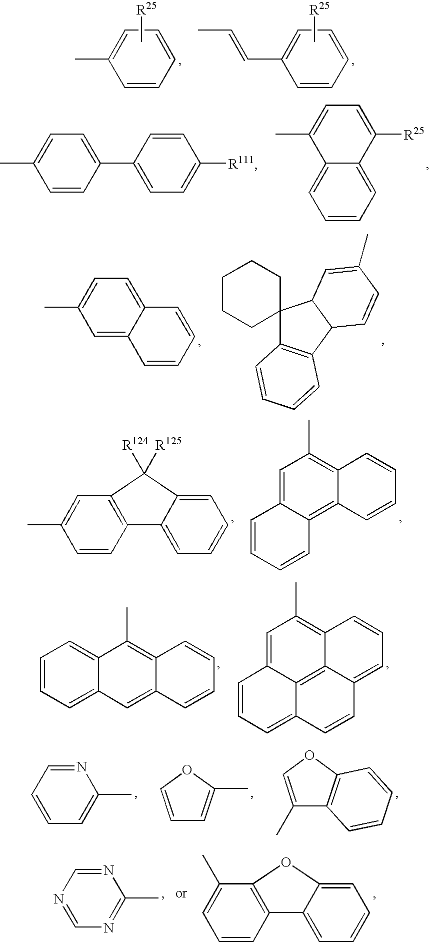

wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl;
Y stand for —CO—NR⁸R⁹, wherein
R⁸ and R⁹ are independently of each other hydrogen, C₁-C₂₅alkyl, phenyl, which can be substituted one to three times with C₁-C₈-alkyl, or

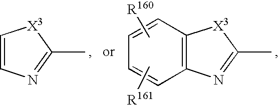

wherein X³ is O, or NR¹⁶³, wherein R¹⁶³ is a C₁-C₂₆alkyl group, which might be interrupted by —O—, a cycloalkyl group, or a phenyl group, and
R¹⁶⁰ and R¹¹ may be the same or different and are selected from hydrogen, or C₁-C₂₅alkyl; or
R⁸ and R⁹ together are C₂-C₈alkylene optionally substituted by C₁-C₈alkyl and/or optionally interrupted by —O—, —S— or —NR¹⁶³—, especially

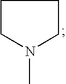

with the proviso that at least one, preferably two of R², R³, R^{2′} and R^{3′} are different from hydrogen.

The following compound of formula VIIi is most preferred:

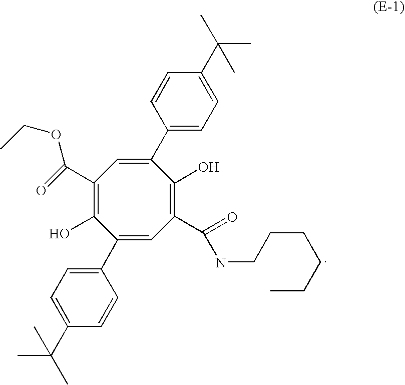

In another embodiment of the present invention compounds of formula

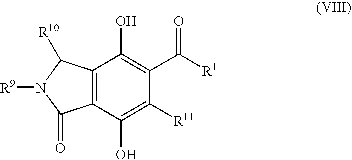

are preferred, wherein
R¹ is —OH, aralkyloxy, especially benzyloxy, which can be substituted one to three times with C₁-C₈alkyl, aryloxy, especially phenoxy, which can be substituted one to three times with C₁-C₈alkyl, or C₁-C₁₈alkoxy,
R⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³, wherein
R³³ and R³⁴ independently from each other stand for hydrogen or C₁-C₄alkyl, or phenyl, which can be substituted one to three times with C₁-C₄alkyl,
A³ stands for aryl or heteroaryl, in particular phenyl or 1- or 2-naphthyl, which can be substituted one to three times with C₁-C₈alkyl and/or C₁-C₈alkoxy, and s stands for 0, 1, 2, 3 or 4,
R¹⁰ stands for aryl or heteroaryl, which can be substituted one to three times with C₁-C₈alkyl and/or C₁-C₈alkoxy,
R¹¹ is H, C₁-C₈alkyl, which optionally forms 5-, 6- or 7-membered rings with R¹ and can optionally be substituted by aryl groups, especially phenyl, which can be optionally substituted by C₁-C₈alkyl.

If R¹⁰ stands for “aryl”, the term “aryl group” is typically C₆-C₂₄aryl, such as phenyl, indenyl, azulenyl, naphthyl, biphenyl, as-indacenyl, s-indacenyl, acenaphthylenyl, fluorenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Preferred examples of C₆-C₁₄aryl are phenyl, 1-naphthyl, 2-naphthyl, 3- or 4-biphenyl, 2- or 9-fluorenyl or 9-phenanthryl, which may be unsubstituted or substituted.

If R¹⁰ stands for “aryl”, it is especially a group of formula

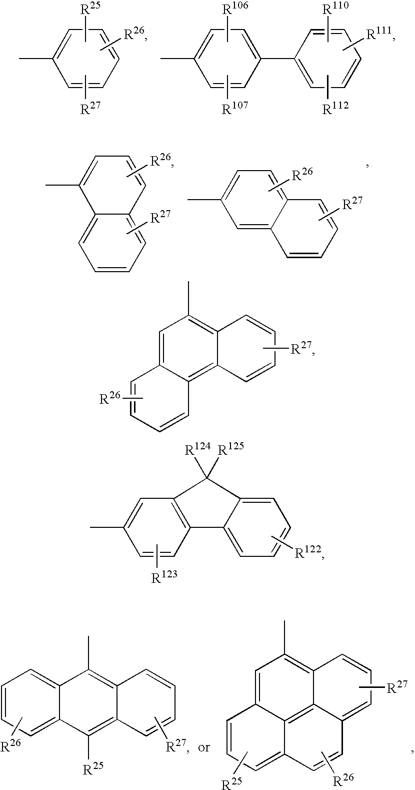

wherein
R¹⁰⁶, R¹⁰⁷, R¹¹⁰, R¹¹¹, R¹¹², R¹²² and R¹²³ are independently of each other hydrogen, C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group or a siloxanyl group;
R¹²⁴ and R¹²⁵ may be the same or different and are selected from C₁-C₁₈alkyl; or R¹²⁴ and R¹²⁵ together form a ring especially a five- or six-membered ring, which can optionally be substituted by C₁-C₈alkyl;
R²⁵, R²⁶, R²⁷ independently from each other stands for hydrogen, C₁-C₂₅-alkyl, —CR²⁸R²⁹—(CH₂)ₙ-A⁶, cyano, halogen, —OR³⁰, —S(O)ₚR³¹, or phenyl, which can be substituted one to three times with C₁-C₈alkyl or C₁-C₃alkoxy, wherein R³⁰ stands for C₁-C₂₅-alkyl, C₅-C₁₂-cycloalkyl, —CR²⁸R²⁹—(CH₂)ᵥ-Ph, C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R³¹ stands for C₁-C₂₅-alkyl, C₅-C₁₂-cycloalkyl, —CR²⁸R²⁹—(CH₂)ᵥ-Ph, R²⁸ and R²⁹ stand for hydrogen, A⁶ stands for phenyl or 1- or 2-naphthyl, which can be substituted one to three times with C₁-C₈alkyl and/or C₁-C₈alkoxy, p stands for 0, 1, 2 or 3, n and v stands for 0, 1, 2, 3 or 4, very especially a group of formula

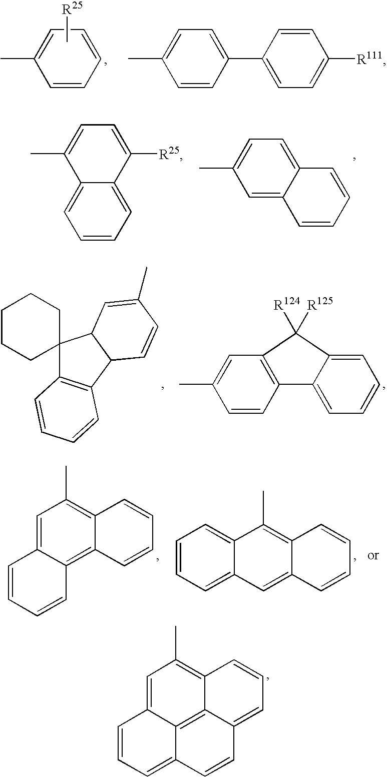

wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl.

If R¹⁰ stands for “heteroaryl”, the term “heteroaryl” means a ring, wherein nitrogen, oxygen or sulfur are the possible hetero atoms, and is typically an unsaturated heterocyclic radical with five to 24 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono-, bi, or tricyclic heterocyclic radicals, which may be unsubstituted or substituted, especially by one to three C₁-C₈alkyl groups.

If R¹⁰ stands for “heteroaryl”, it is especially a group of formula

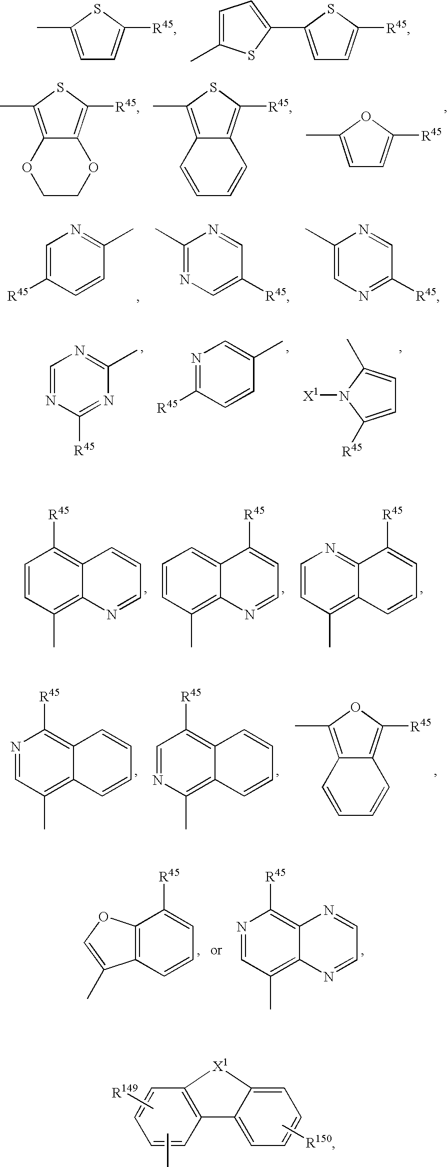

especially

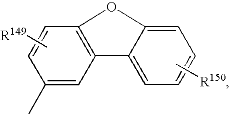

or

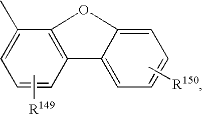

wherein X¹ is O, S, or NR¹⁵³; R¹⁴⁹ and R¹⁵⁰ may be the same or different and are selected from hydrogen, C₁-C₂₅alkyl group, cycloalkyl, aralkyl, alkenyl, cycloalkenyl, alkynyl, hydroxyl, a mercapto group, alkoxy, alkylthio, an aryl ether group, an aryl thioether group, aryl, a heterocyclic group, halogen, haloalkyl, haloalkenyl, haloalkynyl, a cyano group, an aldehyde group, a carbonyl group, a carboxyl group, an ester group, a carbamoyl group, a group NR¹²⁷R¹²⁸, wherein R¹²⁷ and R¹²⁸ independently of each other stand for a hydrogen atom, an alkyl group, an optionally substituted cycloalkyl group, an optionally substituted aryl group, an optionally substituted heteroaryl group, an optionally substituted heterocyclic group, an aralkyl group, or R¹²⁷ and R¹²⁸ together with the nitrogen atom to which they are bonded form a five or six membered heterocyclic ring, which can be condensed by one or two optionally substituted phenyl groups; a nitro group, a silyl group, a siloxanyl group, a substituted or unsubstituted vinyl group,
R¹⁵³ is a hydrogen atom, a C₁-C₂₅alkyl group, which might be interrupted by —O—, a cycloalkyl group, an aralkyl group, an aryl group, or a heterocyclic group,
R⁴⁵ is a hydrogen atom, a C₁-C₁₂alkyl group, a C₁-C₈alkoxy group, a group of formula

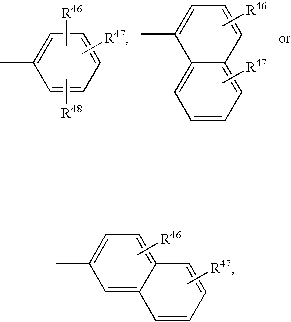

wherein R⁴⁶, R⁴⁷ and R⁴³ independently from each other stands for hydrogen, C₁-C₈-alkyl, C₁-C₈-alkoxy, or phenyl, in particular

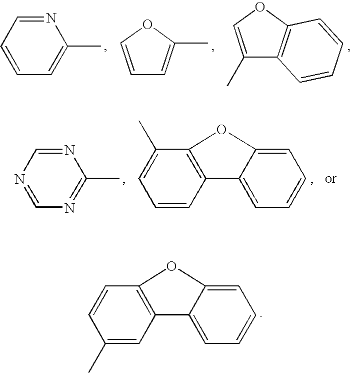

Compounds of the formula (VIII), wherein

R¹⁰ is C₁-C₈alkyl; aryl, or heteroaryl, especially phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, halogen, or —OR⁶;
R¹ is —OH, phenoxy, which can optionally be substituted by one to three C₁-C₈ alkyl groups, or C₁-C₁₈alkoxy;
R⁹ is C₁-C₁₈alkyl, an aryl group, especially phenyl, 1- or 2-naphthyl, an aralkyl group, especially benzyl, or C₅-C₈cycloalkyl, especially cyclohexyl, which can be substituted by one or more groups C₁-C₈alkyl,
R⁶ is C₁-C₈alkyl,
R¹¹ is H, C₁-C₈alkyl, C₆-C₁₄aryl, especially phenyl, 1- or 2-naphthyl, which can optionally be substituted by one to three C₁-C₈alkyl groups, or R¹¹ forms 5-, 6- or 7-membered rings with R¹ and can optionally be substituted by aryl, or heteroaryl groups, especially phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, are especially preferred.

In a preferred embodiment of the present invention R¹⁰, R¹⁰⁰, and R¹¹ in formula

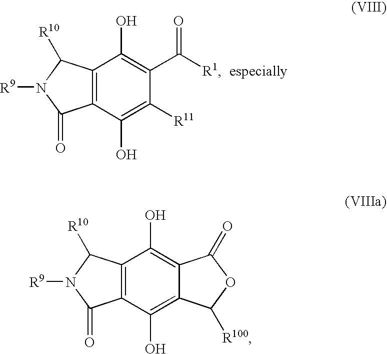

are aryl groups, or heteroaryl groups, wherein R¹⁰ and R¹¹ are different from each other and R¹⁰ and R¹⁰⁰ are different from each other, respectively; or R¹⁰ is an aryl group, or heteroaryl group, and R¹ and R¹¹ are a group —CHR¹⁰¹CHR¹⁰²—O—, wherein R¹⁰¹ and R¹⁰² are as defined above, —CR¹⁰³═CR¹⁰⁴—O—, wherein R¹⁰³ and R¹⁰⁴ are as defined above.

Preferred are compounds of for (VIII), wherein

R¹ is —OH, C₇-C₃₀aralkyloxy, especially benzyloxy, which can be substituted one to three times with C₁-C₈-alkyl, or C₁-C₈-alkoxy, C₆-C₂₄aryloxy, especially phenoxy, which can be substituted one to three times with C₁-C₈-alkyl, or C₁-C₈alkoxy,
R⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³, wherein
R³³ and R³⁴ independently from each other stand for hydrogen or C₁-C₄alkyl, or phenyl, which can be substituted one to three times with C₁-C₄alkyl,
A³ stands for aryl or heteroaryl, in particular phenyl or 1- or 2-naphthyl, which can be substituted one to three times with C₁-C₈alkyl and/or C₁-C₈alkoxy, and s stands for 0, 1, 2, 3 or 4,
R¹⁰ stands for aryl or heteroaryl, which can be substituted one to three times with C₁-C₈alkyl and/or C₁-C₈alkoxy,
R¹¹ is H, C₁-C₈alkyl, C₆-C₁₄aryl, especially phenyl, or naphthyl, which can be optionally substituted by one or more C₁-C₈alkyl groups, or
R¹¹ forms 5-, 6- or 7-membered rings with R¹, which can optionally be substituted by aryl, or heteroaryl groups, especially phenyl, biphenyl, naphthyl, fluorenyl, anthryl, pyrenyl, phenanthryl, pyridyl, furanyl, benzofuranyl, triazinyl, or dibenzofuranyl, which can be substituted one to three times with C₁-C₈-alkyl; or which can be condensed one time by phenyl which can be substituted one to three times with C₁-C₄-alkyl.

Especially preferred are compounds of formula VIII, wherein

R¹⁰ is H, C₁-C₈alkyl;

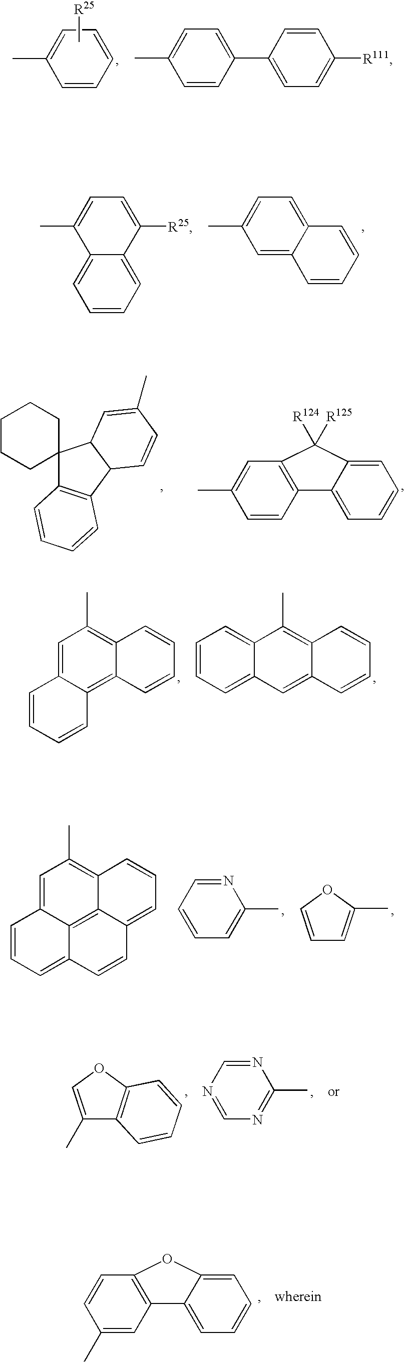

R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl, which can optionally be interrupted by —O—;
R¹ is —OH, benzyloxy, which can be substituted one to three times with C₁-C₈-alkyl, phenoxy, which can be substituted one to three times with C₁-C₈-alkyl, or C₁-C₁₈alkoxy,
R⁹ is C₁-C₁₈alkyl, an aryl group, especially phenyl, 1- or 2-naphthyl, which can be substituted by one or more groups C₁-C₈alkyl, or C₅-C₈cycloalkyl, which can be substituted by one or more groups C₁-C₈alkyl,
R¹¹ is H, C₁-C₈alkyl, C₆-C₁₄aryl, especially phenyl, or naphthyl, which can be optionally substituted by one or more C₁-C₈alkyl groups, or
R¹¹ and R¹ together form a ring and are a group —CHR¹⁰⁰—O—, wherein R¹⁰⁰ is hydrogen, C₁-C₁₈alkyl, C₆-C₂₄aryl, especially phenyl, biphenyl, naphthyl, fluorenyl, or phenanthryl, which can be substituted one to three times with C₁-C₈-alkyl, or
R¹¹ and R¹ together form a ring and are a group —CHR¹⁰¹CHR¹⁰²—O—, wherein R¹⁰¹ and R¹⁰² are independently of each other hydrogen, C₁-C₁₈alkyl, C₆-C₂₄aryl, especially phenyl, biphenyl, naphthyl, fluorenyl, or phenanthryl, which can be substituted one to three times with C₁-C₈-alkyl, or
R¹¹ and R¹ together form a ring and are a group —CR¹⁰³═CR¹⁰⁴—O—, wherein R¹⁰³ and R¹⁰⁴ are independently of each other hydrogen, C₁-C₁₈alkyl, C₆-C₂₄aryl, especially phenyl, biphenyl, naphthyl, fluorenyl, or phenanthryl, which can be substituted one to three times with C₁-C₈-alkyl, or
R¹⁰³ and R¹⁰⁴ together form a ring and are a group

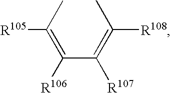

wherein R¹⁰⁵, R¹⁰⁶, R¹⁰⁸ and R¹⁰⁹ are independently of each other hydrogen, or C₁-C₁₈alkyl.

The following compounds of formula (VIII) are most preferred:

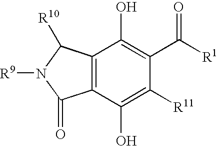

| Compound | R¹ | R¹¹ | R⁹ | R¹⁰ |
|---|---|---|---|---|
| A-1 | —O-i-C₃H₇ | H | CH₃ | 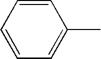 |
| A-2 | —O-i-C₃H₇ | H | CH₃ | 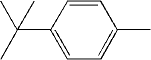 |
| A-3 | —O-i-C₃H₇ | H | CH₃ | 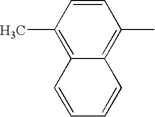 |
| A-4 | —O-tBu | H | n-C₃H₇ | 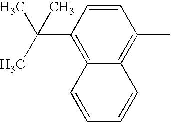 |
| A-5 | —O-tBu | H | n-C₃H₇ | 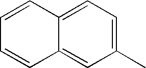 |
| A-6 | —O-tBu | H | C₂H₅ | 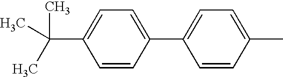 |
| A-7 | —O-i-C₃H₇ | H | CH₃ | 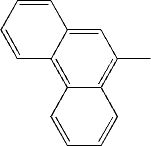 |
| A-8 | —O-tBu | H | CH₃ | 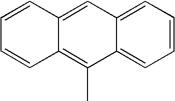 |
| A-9 | —O-n-C₉H₂₁ | H | 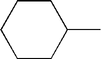 | 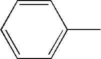 |
| A-10 | 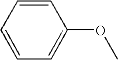 | H | 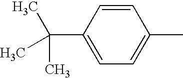 | 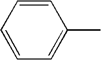 |
| A-11 | ¹⁾ | ¹⁾ | CH₃ | 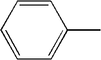 |
| A-12 | ²⁾ | ²⁾ | CH₃ | 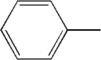 |
| A-13 | ³⁾ | ³⁾ | CH₃ | 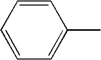 |
| A-14 | ¹⁾ | ¹⁾ | CH₃ | 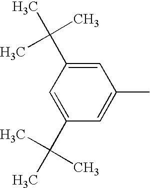 |
| A-15 | ¹⁾ | ¹⁾ | CH₃ | 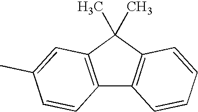 |
| A-16 | —O—CH₃ | 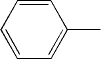 | CH₃ | 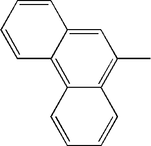 |
| A-17 | ⁴⁾ | ⁴⁾ | CH₃ | 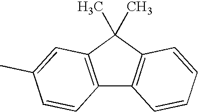 |
| A-18 | —O—CH₃ | 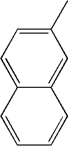 | CH₃ | 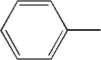 |
| A-19 | ⁵⁾ | ⁵⁾ | CH₃ | 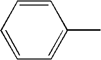 |
| A-20 | ⁵⁾ | ⁵⁾ | CH₃ | 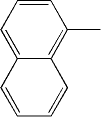 |
| A-21 | ⁶⁾ | ⁶⁾ | CH₃ | 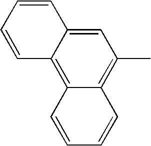 |
| A-22 | ⁷⁾ | ⁷⁾ | CH₃ | 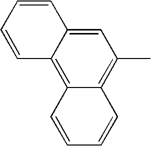 |
| A-23 | ⁵⁾ | ⁵⁾ | CH₃ | 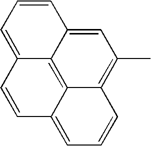 |
| A-24 | ⁵⁾ | ⁵⁾ | CH₃ | 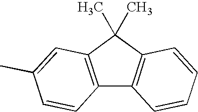 |
| A-25 | ⁸⁾ | ⁸⁾ | CH₃ | 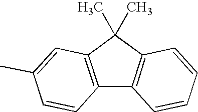 |
| A-26 | ⁴⁾ | ⁴⁾ | CH₃ | 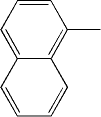 |
| A-27 | —O-i-C₃H₇ | —O-i-C₃H₇ | n-C₃H₇ | 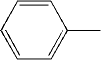 |
| A-28 | —O-i-C₃H₇ | H | n-propyl | phenyl |
| A-29 | —O-i-C₃H₇ | H | n-propyl | methyl |
| A-30 | —O-i-C₃H₇ | H | H | 1-naphthyl |
| A-31 | —O-i-C₃H₇ | H | H | 9-phenanthrenyl |
| A-32 | —O-i-C₃H₇ | H | H | phenyl |
| A-33 | —O-i-C₃H₇ | H | H | methyl |
| A-34 | —O-i-C₃H₇ | H | H | 4-bromo-phenyl |
| A-35 | —O-i-C₃H₇ | H | H | 4-methyl-phenyl |
| A-36 | —O-i-C₃H₇ | H | H | 2-methoxy-phenyl |
| A-37 | —O-i-C₃H₇ | H | H | 4-methyl-1-naphthyl |
| B-1 | —O—C₂H₅ | H | i-C₃H₇ | 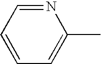 |
| B-2 | —O-i-C₃H₇ | H | tBu | 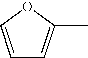 |
| B-3 | —O-i-C₃H₇ | H | CH₃ | 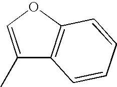 |
| B-4 | —O-i-C₃H₇ | H | CH₃ | 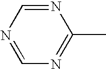 |
| B-5 | ¹⁾ | ¹⁾ | CH₃ | 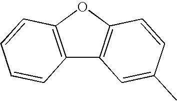 |
| B-6 | ⁵⁾ | ⁵⁾ | CH₃ | 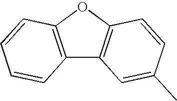 |

¹⁾R¹¹ and R¹ together form a ring and are a group —CH₂—O—.
²⁾R¹¹ and R¹ together form a ring and are a group —CH₂CH₂—O—.
³⁾R¹¹ and R¹ together form a ring and are a group —CH═CH—O—.

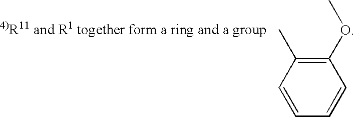

⁵⁾R¹¹ and R¹ together form a ring and are a group —CH(Ph)—O—.

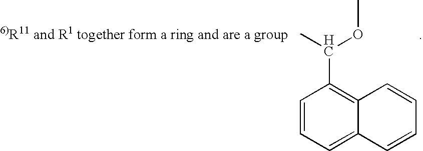

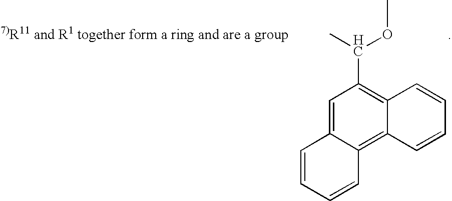

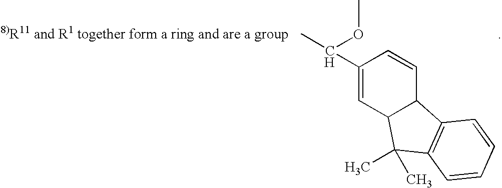

The term “halogen” means fluorine, chlorine, bromine and iodine.

C₁-C₂₆alkyl is typically linear or branched—where possible—methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl, n-nonyl, decyl, undecyl, dodecyl, tetradecyl, pentadecyl, hexadecyl, heptadecyl, octadecyl, eicosyl, heneicosyl, docosyl, tetracosyl or pentacosyl, preferably C₁-C₈alkyl such as methyl, ethyl, n-propyl, isopropyl, n-butyl, sec.-butyl, isobutyl, tert.-butyl, n-pentyl, 2-pentyl, 3-pentyl, 2,2-dimethyl-propyl, n-hexyl, n-heptyl, n-octyl, 1,1,3,3-tetramethylbutyl and 2-ethylhexyl, more preferably C₁-C₄alkyl such as typically methyl, ethyl, n-propyl, isopropyl, n-butyl, sec.-butyl, isobutyl, tert.-butyl.

The terms “haloalkyl, haloalkenyl and haloalkynyl” mean groups given by partially or wholly substituting the above-mentioned alkyl group, alkenyl group and alkynyl group with halogen, such as trifluoromethyl etc. The “aldehyde group, ketone group, ester group, carbamoyl group and amino group” include those substituted by an alkyl group, a cycloalkyl group, an aryl group, an aralkyl group or a heterocyclic group, wherein the alkyl group, the cycloalkyl group, the aryl group, the aralkyl group and the heterocyclic group may be unsubstituted or substituted. The term “silyl group” means a group of formula —SiR⁶²R⁶³R⁶⁴, wherein R⁶², R⁶³ and R⁶⁴ are independently of each other a C₁-C₈alkyl group, in particular a C₁-C₄ alkyl group, a C₆-C₂₄aryl group or a C₇-C₁₂aralkyl group, such as a trimethylsilyl group. The term “siloxanyl group” means a group of formula —O—SiR⁶²R⁶³R⁶⁴, wherein R⁶², R⁶³ and R⁶⁴ are as defined above, such as a trimethylsiloxanyl group.

Examples of C₁-C₁₈alkoxy, especially C₁-C₈alkoxy are methoxy, ethoxy, n-propoxy, isopropoxy, n-butoxy, sec.-butoxy, isobutoxy, tert.-butoxy, n-pentoxy, 2-pentoxy, 3-pentoxy, 2,2-dimethylpropoxy, n-hexoxy, n-heptoxy, n-octoxy, 1,1,3,3-tetramethylbutoxy and 2-ethylhexoxy, preferably C₁-C₄alkoxy such as typically methoxy, ethoxy, n-propoxy, isopropoxy, n-butoxy, sec.-butoxy, isobutoxy, tert.-butoxy. The term “alkylthio group” means the same groups as the alkoxy groups, except that the oxygen atom of ether linkage is replaced by a sulfur atom.

The term “aryl group” is typically C₆-C₂₄aryl, such as phenyl, indenyl, azulenyl, naphthyl, biphenyl, as-indacenyl, s-indacenyl, acenaphthylenyl, phenanthryl, fluoranthenyl, triphenlenyl, chrysenyl, naphthacen, picenyl, perylenyl, pentaphenyl, hexacenyl, pyrenyl, or anthracenyl, preferably phenyl, 1-naphthyl, 2-naphthyl, 9-phenanthryl, 2- or 9-fluorenyl, 3- or 4-biphenyl, which may be unsubstituted or substituted. Examples of C₆-C₁₂aryl are phenyl, 1-naphthyl, 2-naphthyl, 3- or 4-biphenyl, which may be unsubstituted or substituted.

The term “aralkyl group” is typically C₇-C₂₄aralkyl, such as benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ω-phenyl-butyl, ω,ωdimethyl-ω-phenyl-butyl, ω-phenyl-dodecyl or ω-phenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted.

The term “aryl ether group” is typically a C₆₋₂₄aryloxy group, that is to say O—C₆₋₂₄aryl, such as, for example, phenoxy or 4-methoxyphenyl. The term “aryl thioether group” is typically a C₆₋₂₄arylthio group, that is to say S—C₆₋₂₄aryl, such as, for example, phenylthio or 4-methoxyphenylthio. The term “carbamoyl group” is typically a C₁₋₁₈carbamoyl radical, preferably C₁₋₈carbamoyl radical, which may be unsubstituted or substituted, such as, for example, carbamoyl, methylcarbamoyl, ethylcarbamoyl, n-butylcarbamoyl, tert-butylcarbamoyl, dimethylcarbamoyloxy, morpholinocarbamoyl or pyrrolidinocarbamoyl.

The term “cycloalkyl group” is typically C₅-C₁₂cycloalkyl, such as cyclopentyl, cyclohexyl, cycloheptyl, cyclooctyl, cyclononyl, cyclodecyl, cycloundecyl, cyclodcdecyl, preferably cyclopentyl, cyclohexyl, cycloheptyl, or cyclooctyl, which may be unsubstituted or substituted. The term “cycloalkenyl group” means an unsaturated alicyclic hydrocarbon group containing one or more double bonds, such as cyclopentenyl, cyclopentadienyl, cyclohexenyl and the like, which may be unsubstituted or substituted. The cycloalkyl group, in particular a cyclohexyl group, can be condensed one or two times by phenyl which can be substituted one to three times with C₁-C₄-alkyl, halogen and cyano. Examples of such condensed cyclohexyl groups are:

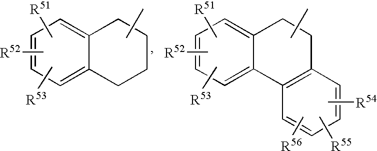

or

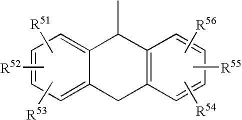

in particular

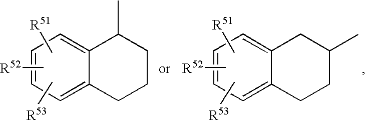

wherein R⁵¹, R⁵², R⁵³, R⁵⁴, R⁵⁵ and R⁵⁶ are independently of each other C₁-C₈-alkyl, C₁-C₈-alkoxy, halogen and cyano, in particular hydrogen.

The term “heteroaryl group” is a ring, wherein nitrogen, oxygen or sulfur are the possible hetero atoms, and is typically an unsaturated heterocyclic radical with five to 24 atoms having at least six conjugated n-electrons such as thienyl, benzo[b]thienyl, dibenzo[b,d]thienyl, thianthrenyl, furyl, furfuryl, 2H-pyranyl, benzofuranyl, isobenzofuranyl, 2H-chromenyl, xanthenyl, dibenzofuranyl, phenoxythienyl, pyrrolyl, imidazolyl, pyrazolyl, pyridyl, bipyridyl, triazinyl, pyrimidinyl, pyrazinyl, 1H-pyrrolizinyl, isoindolyl, pyridazinyl, indolizinyl, isoindolyl, indolyl, 3H-indolyl, phthalazinyl, naphthyridinyl, quinoxalinyl, quinazolinyl, cinnolinyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, 4aH-carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocyclic radicals, which may be unsubstituted or substituted.

The terms “aryl” and “alkyl” in alkylamino groups, dialkylamino groups, alkylarylamino groups, arylamino groups and diaryl groups are typically C₁-C₂₅alkyl and C₈-C₂₄aryl, respectively.

The above-mentioned groups can be substituted by a C₁-C₈alkyl, a hydroxyl group, a mercapto group, C₁-C₈alkoxy, C₁-C₈alkylthio, halogen, halo-C₁-C₈allyl, a cyano group, an aldehyde group, a ketone group, a carboxyl group, an ester group, a carbamoyl group, an amino group, a nitro group, a silyl group or a siloxanyl group.

Examples of 5-, 6- or 7-membered rings formed by R¹ and R², or R³ are rings of formula

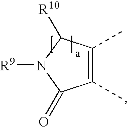

wherein R⁹ and R¹⁰ are as defined above and a is an integer 1, 2, or 3, especially 1.

Examples of 5-, 6- or 7-membered rings formed by R² and R³ are rings of formula

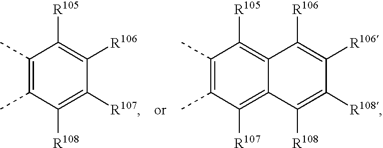

wherein R¹⁰⁵, R¹⁰⁶, R¹⁰⁷, R¹⁰⁸, R^{106′} and R^{108′} are independently of each other H, C₁-C₁₈alkyl, or C₁-C₁₈alkoxy.

Examples of 5-, 6- or 7-membered rings formed by R⁴ and R⁵ are cyclopentane, cyclohexane and cycloheptane, which can optionally be substituted by halogen, —OH, C₁-C₁₈alkyl, or C₁-C₁₈alkoxy.

Examples of 5-, 6- or 7-membered rings formed by R¹, R², R³, R⁴, or R⁵ and X, Y¹, or Y² are rings of formula

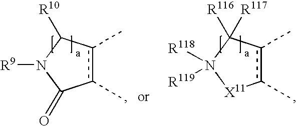

wherein R⁹ is as defined above, R¹¹⁶, R¹¹⁷, R¹¹⁸ and R¹¹⁹ are independently of each other H, C₁-C₁₈alkyl, or C₁-C₁₈alkoxy, or R¹¹⁶ and R¹¹⁷ and/or R¹¹⁸ and R¹¹⁹ together are a group C═O, X¹¹ is —O—, —S—, or —N—R⁹ and a is an integer 1, 2, or 3.

The compounds of formula I can be prepared according to or in analogy to processes known in the art (see, for example, U.S. Pat. No. 4,778,899 and U.S. Pat. No. 4,659,775).

The compounds of formula VIII are, for example, obtainable by reacting the pyrrolinone compounds of formula IX with a compound RO(O)—CH(R¹¹)—CH₂—C(O)—R¹ in the presence of a strong base.

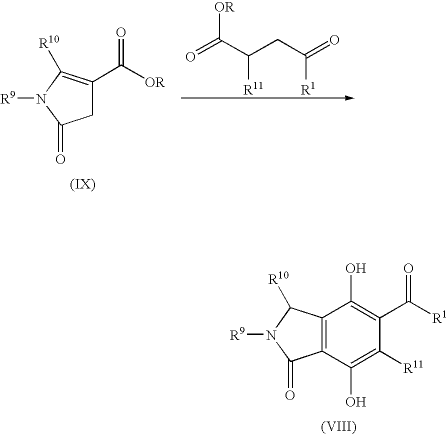

The pyrrolinone compounds of formula IX are, for example, obtained by reacting a compound of formula X with an amine NH₂R⁹.

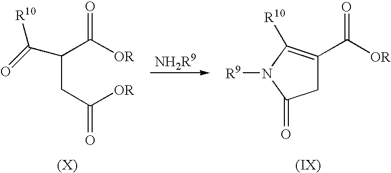

The compounds of formula X are prepared, for example, by condensing an acylacetate of formula XII with an ester of formula XIII, wherein Y is halogen, especially chlorine and bromine (see, for example, U.S. Pat. No. 4,778,899 and U.S. Pat. No. 4,659,775).

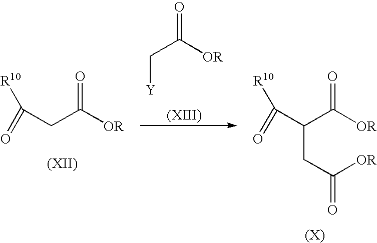

The fluorescent materials according to the present invention can be used for coloring high molecular weight organic materials, as fluorescent tracers, in solid dye lasers, EL lasers, in EL devices and for lighting.

For use in EL lasers and in EL devices they are preferably used as guests in combination with host compounds.

Accordingly the present invention relates also to a composition comprising a guest chromophore and a host chromophore, wherein the absorption spectrum of the guest chromophore overlaps with the fluorescence emission spectrum of the host chromophore, wherein the host chromophore is a fluorescent compound having a photoluminescence emission peak at 300 to 550 nm, preferably 330 to 500 nm, most preferred 360 to 430 nm and wherein the guest chromophore is a compound of formula I.

Suitable host materials are, for example, described in WO2004/020372 and JP2003/192684. Especially suitable are the derivatives of structure XIII, i.e. compounds AA-52 to AA-82, described in WO2004/020372 and CBP and DPVBi described in JP2003/192684:

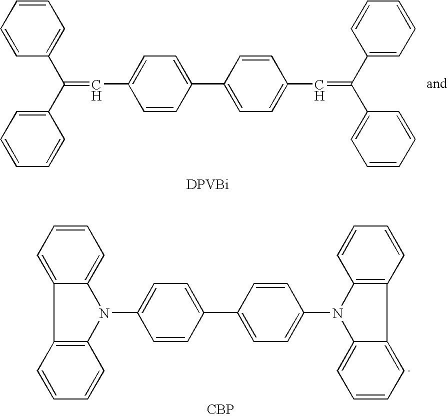

Preferred host chromophores are represented by the formulae below:

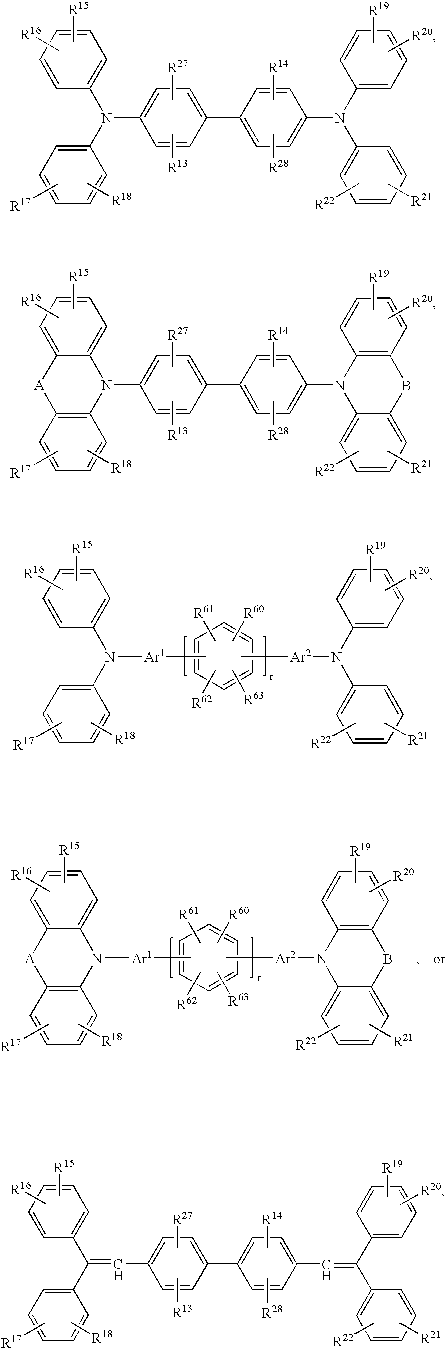

wherein r is an integer 1 to 10,
R¹³, R¹⁴, R¹⁵, R¹⁶, R¹⁷, R¹⁸, R¹⁹, R²⁰, R²¹, R²², R²⁷, R²⁸, R⁶⁰, R⁶¹, R⁶³ are independently of each other hydrogen, halogen, —CN, or C₁-C₁₂alkyl, which can optionally be substituted by one or more groups C₁-C₈alkyl or halogen; phenyl, naphthyl, or phenanthryl which can optionally be substituted by one or more groups C₁-C₈alkyl, halogen, —OR²³ or —NR²⁵R²⁶; —OR²³, —SR²⁴ or —NR²⁵R²⁶; wherein the substituents R²³, R²⁴, R²⁵, or R²⁶ optionally form 5-, 6- or 7-membered rings with adjacent substituents;
R²³ and R²⁴ are hydrogen, C₁-C₁₂alkyl or phenyl;
R²⁵ and R²⁶ are independently of each other hydrogen, C₁-C₂₀alkyl; or R²⁵ and R²⁶ together are C₂-C₈alkylene optionally substituted by C₁-C₈alkyl and/or optionally interrupted by —O—, —S— or —N—,
A and B are independently of each other a direct bond, —CHR¹³—, —CHR¹³CHR¹⁴—, —NR²⁵—, —O—, or —S—,
Ar¹ and Ar² are identical or different from each other and are a substituted or unsubstituted C₆-C₂₄ aromatic or a substituted or unsubstituted C₁-C₂₄ hetero aromatic group, especially phenyl, naphthyl, or phenanthryl, which can optionally be substituted by one or more groups C₁-C₈alkyl, halogen, —OR²³, —SR²⁴, or —NR²⁵R²⁶.

The weight ratio of the host chromophore to the guest chromophore is in general 50:50 to 99.99:0.01, preferably 80:20 to 99.99:0.01, more preferably 90:10 to 99.9:0.1, most preferably 95:5 to 99.9:0.1.

The present invention relates further to an electroluminescent device having the fluorescent compounds of formula I or the compositions according to the present invention between an anode and a cathode and emitting light by the action of electrical energy. The EL device exhibits properties sufficient for practical applications, i.e. a high efficiency and a long life. The organic EL devices of the present invention, which emits white light, can be used as full color display, when color filters or color changing media are attached to a display apparatus.

Typical constitutions of latest organic electroluminescent devices are:

(i) an anode/a hole transporting layer/an electron transporting layer/a cathode, in which the compounds or compositions of the present invention are used either as positive-hole transport compound or composition, which is exploited to form the light emitting and hole transporting layers, or as electron transport compounds or compositions, which can be exploited to form the light-emitting and electron transporting layers,
(ii) an anode/a hole transporting layer/a light-emitting layer/an electron transporting layer/a cathode, in which the compounds or compositions form the light-emitting layer regardless of whether they exhibit positive-hole or electron transport properties in this constitution,
(iii) an anode/a hole injection layer/a hole transporting layer/a light-emitting layer/an electron transporting layer/a cathode,
(iv) an anode/a hole transporting layer/a light-emitting layer/a hole-blocking layer/an electron transporting layer/a cathode,
(v) an anode/a hole injection layer/a hole transporting layer/a light-emitting layer/a hole-blocking layer/an electron transporting layer/a cathode,
(vi) an anode/a light-emitting layer/an electron transporting layer/a cathode,
(vii) an anode/a light-emitting layer/a hole-blocking layer/an electron transporting layer/a cathode,
(viii) a mono-layer containing a light emitting material alone or a combination a light emitting material and any of materials of the hole transporting layer, the hole-blocking layer and/or the electron transporting layer, and
(ix) a multi-layered structure described in (ii) to (vii), wherein a light emitting layer is the mono-layer defined in (viii).

The compounds and compositions of the present invention can, in principal, be used for any organic layer, such as, for example, hole transporting layer, light emitting layer, or electron transporting layer, but are preferably used as the light emitting material in the light emitting layer.

Thin film type electroluminescent devices usually consist essentially of a pair of electrodes and at least one charge transporting layer in between. Usually two charge transporting layers, a hole transporting layer (next to the anode) and an electron transporting layer (next to the cathode) are present. Either one of them contains—depending on its properties as hole-transporting or electron-transporting material—an inorganic or organic fluorescence substance as light-emitting material. It is also common, that a light-emitting material is used as an additional layer between the hole-transporting and the electron-transporting layer. In the above mentioned device structure, a hole injection layer can be constructed between an anode and a hole transporting layer and/or a hole blocking layer can be constructed between a light emitting layer and an electron transporting layer to maximise hole and electron population in the light emitting layer, reaching large efficiency in charge recombination and intensive light emission.

The devices can be prepared in several ways. Usually, vacuum evaporation is used for the preparation. Preferably, the organic layers are laminated in the above order on a commercially available indium-in-oxide (“ITO”) glass substrate held at room temperature, which works as the anode in the above constitutions. The membrane thickness is preferably in the range of 1 to 10,000 nm, more preferably 1 to 5,000 nm, more preferably 1 to 1,000 nm, more preferably 1 to 500 nm. The cathode metal, such as a Mg/Ag alloy, a binary Li—Al or LiF—Al system with an thickness in the range of 50-200 nm is laminated on the top of the organic layers. The vacuum during the deposition is preferably less than 0.1333 Pa (1×10⁻³ Torr), more preferably less than 1.333×10⁻³ Pa (1×10⁻⁵ Torr), more preferably less than 1.333×10⁻⁴ Pa (1×10⁻⁶ Torr).

As anode usual anode materials which possess high work function such as metals like gold, silver, copper, aluminum, indium, iron, zinc, tin, chromium, titanium, vanadium, cobalt, nickel, lead, manganese, tungsten and the like, metallic alloys such as magnesium/copper, magnesium/silver, magnesium/aluminum, aluminum/indium and the like, semiconductors such as Si, Ge, GaAs and the like, metallic oxides such as indium-tin-oxide (“ITO”), indium-zinc-oxide (IZO), ZnO and the like, metallic compounds such as CuI and the like, and furthermore, electroconducting polymers such polyacetylene, polyaniline, polythiophene, polypyrrole, polyparaphenylene and the like, preferably ITO, most preferably ITO on glass as substrate can be used.

Of these electrode materials, metals, metallic alloys, metallic oxides and metallic compounds can be transformed into electrodes, for example, by means of the sputtering method. In the case of using a metal or a metallic alloy as a material for an electrode, the electrode can be formed also by the vacuum deposition method. In the case of using a metal or a metallic alloy as a material forming an electrode, the electrode can be formed, furthermore, by the chemical plating method (see for example, Handbook of Electrochemistry, pp 383-387, Mazuren, 1985). In the case of using an electroconducting polymer, an electrode can be made by forming it into a film by means of anodic oxidation polymerization method onto a substrate which is previously provided with an electroconducting coating. The thickness of an electrode to be formed on a substrate is not limited to a particular value, but, when the substrate is used as a light emitting plane, the thickness of the electrode is preferably within the range of from 1 nm to 300 nm, more preferably, within the range of from 5 to 200 nm so as to ensure transparency.

In a preferred embodiment ITO is used on a substrate having an ITO film thickness in the range of from 10 nm (100 Å) to 1μ (10000 Å), preferably from 20 nm (200 Å) to 500 nm (5000 Å). Generally, the sheet resistance of the ITO film is chosen in the range of not more than 100 Ω/cm², preferably not more than 50 Ω/cm². Such anodes are commercially available from Japanese manufacturers, such as Geomatech Co. Ltd., Sanyo Vacuum Co. Ltd., Nippon Sheet Glass Co. Ltd.

As substrate either an electroconducting or electrically insulating material can be used. In case of using an electroconducting substrate, a light emitting layer or a positive hole transporting layer is directly formed thereupon, while in case of using an electrically insulating substrate, an electrode is firstly formed thereupon and then a light emitting layer or a positive hole transporting layer is superposed.

The substrate may be either transparent, semi-transparent or opaque. However, in case of using a substrate as an indicating plane, the substrate must be transparent or semi-transparent.

Transparent electrically insulating substrates are, for example, inorganic compounds such as glass, quartz and the like, organic polymeric compounds such as polyethylene, polypropylene, polymethylmethacrylate, polyacrylonitrile, polyester, polycarbonate, polyvinylchloride, polyvinylalcohol, polyvinylacetate and the like. Each of these substrates can be transformed into a transparent electroconducting substrate by providing it with an electrode according to one of the methods described above.

Examples of semi-transparent electrically insulating substrates are inorganic compounds such as alumina, YSZ (yttrium stabilized zirconia) and the like, organic polymeric compounds such as polyethylene, polypropylene, polystyrene, epoxy resins and the like. Each of these substrates can be transformed into a semi-transparent electroconducting substrate by providing it with an electrode according to one of the abovementioned methods.

Examples of opaque electroconducting substrates are metals such as aluminum, indium, iron, nickel, zinc, tin, chromium, titanium, copper, silver, gold, platinum and the like, various electroplated metals, metallic alloys such as bronze, stainless steel and the like, semiconductors such as Si, Ge, GaAs, and the like, electroconducting polymers such as polyaniline, polythiophene, polypyrrole, polyacetylene, polyparaphenylene and the like.

A substrate can be obtained by forming one of the above listed substrate materials to a desired dimension. It is preferred that the substrate has a smooth surface. Even, if it has a rough surface, it will not cause any problem for practical use, provided that it has round unevenness having a curvature of not less than 20 μm. As for the thickness of the substrate, there is no restriction as far as it ensures sufficient mechanical strength.

As cathode usual cathode materials which possess low work function such as alkali metals, earth alkaline metals, group 13 elements, silver, and copper as well as alloys or mixtures thereof such as sodium, lithium, potassium, calcium, lithium fluoride (LiF), sodium-potassium alloy, magnesium, magnesium-silver alloy, magnesium-copper alloy, magnesium-aluminum alloy, magnesium-indium alloy, aluminum, aluminum-aluminum oxide alloy, aluminum-lithium alloy, indium, calcium, and materials exemplified in EP-A 499,011 such as electroconducting polymers e.g. polypyrrole, polythiophene, polyaniline, polyacetylene etc., preferably Mg/Ag alloys, LiF—Al or Li—Al compositions can be used.

In a preferred embodiment a magnesium-silver alloy or a mixture of magnesium and silver, or a lithium-aluminum alloy, lithium fluoride-aluminum alloy or a mixture of lithium and aluminum can be used in a film thickness in the range of from 10 nm (100 Å) to 1 μm (10000 Å), preferably from 20 nm (200 Å) to 500 nm (5000 Å).

Such cathodes can be deposited on the foregoing electron transporting layer by known vacuum deposition techniques described above.

In a preferred embodiment of this invention a light-emitting layer can be used between the hole transporting layer and the electron transporting layer. Usually the light-emitting layer is prepared by forming a thin film on the hole transporting layer.

As methods for forming said thin film, there are, for example, the vacuum deposition method, the spin-coating method, the casting method, the Langmuir-Blodgett (“LB”) method and the like. Among these methods, the vacuum deposition method, the spin-coating method and the casting method are particularly preferred in view of ease of operation and cost.

In case of forming a thin film using a composition by means of the vacuum deposition method, the conditions under which the vacuum deposition is carried out are usually strongly dependent on the properties, shape and crystalline state of the compound(s). However, optimum conditions are usually as follows: temperature of the heating boat: 100 to 400° C.; substrate temperature: −100 to 350° C.; pressure: 1.33×10⁴ Pa (1×10² Torr) to 1.33×10⁻⁴ Pa (1×10⁻⁶ Torr) and deposition rate: 1 pm to 6 nm/sec.

In an organic EL element, the thickness of the light emitting layer is one of the factors determining its light emission properties. For example, if a light emitting layer is not sufficiently thick, a short circuit can occur quite easily between two electrodes sandwiching said light emitting layer, and therefor, no EL emission is obtained. On the other hand, if the light emitting layer is excessively thick, a large potential drop occurs inside the light emitting layer because of its high electrical resistance, so that the threshold voltage for EL emission increases. Accordingly, the thickness of the organic light emitting layer is limited to the range of from 5 nm to 5 μm, preferably to the range of from 10 nm to 500 nm.

In the case of forming a light emitting layer by using the spin-coating method and the casting method, ink jet printing method, the coating can be carried out using a solution prepared by dissolving the composition in a concentration of from 0.0001 to 90% by weight in an appropriate organic solvent such as benzene, toluene, xylene, tetrahydrofurane, methyltetrahydrofurane, N,N-dimethylformamide, dichloromethane, dimethylsulfoxide and the like. If the concentration exceeds 90% by weight, the solution usually is so viscous that it no longer permits forming a smooth and homogenous film. On the other hand, if the concentration is less than 0.0001% by weight, the efficiency of forming a film is too low to be economical. Accordingly, a preferred concentration of the composition is within the range of from 0.01 to 80% by weight.

In the case of using the above spin-coating or casting method, it is possible to further improve the homogeneity and mechanical strength of the resulting layer by adding a polymer binder to the solution for forming the light emitting layer. In principle, any polymer binder may be used, provided that it is soluble in the solvent in which the composition is dissolved. Examples of such polymer binders are polycarbonate, polyvinylalcohol, polymethacrylate, polymethylmethacrylate, polyester, polyvinylacetate, epoxy resin and the like. However, if the solid content composed of the polymer binder and the composition exceeds 99% by weight, the fluidity of the solution is usually so low that it is impossible to form a light emitting layer excellent in homogeneity. On the other hand, if the content of the composition is substantially smaller than that of the polymer binder, the electrical resistance of said layer is very large, so that it does not emit light unless a high voltage is applied thereto. Accordingly, the preferred ratio of the polymer binder to the composition is chosen within the range of from 10:1 to 1:50 by weight, and the solid content composed of both components in the solution is preferably within the range of from 0.01 to 80% by weight, and more preferably, within the range of 0.1 to 60% by weight.

As hole-transporting layers known organic hole transporting compounds such as polyvinyl carbazole

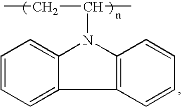

DPA-PS, DPA-PMMA, DPAD-PMMA, TPD-PMMAA, TPD-CPA, polythiophene, polysilane (as shown in WO2004/020372 (page 36) as polymeric hole transporting materials), a TPD compound disclosed in J. Amer. Chem. Soc. 90 (1968) 3925:

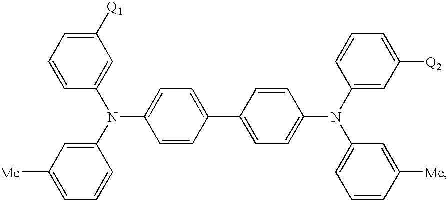

wherein Q₁ and Q₂ each represent a hydrogen atom or a methyl group;

a compound disclosed in J. Appl. Phys. 65(9) (1989) 3610:

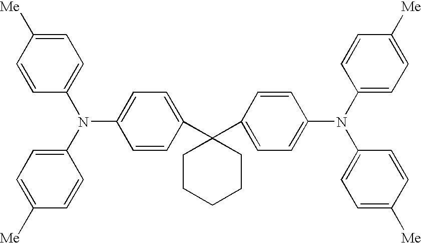

compounds described in WO2004/020372 (page 36), such as m-MTDATA, DTDPFL, spiro-TPD, TPAC, or PDA,

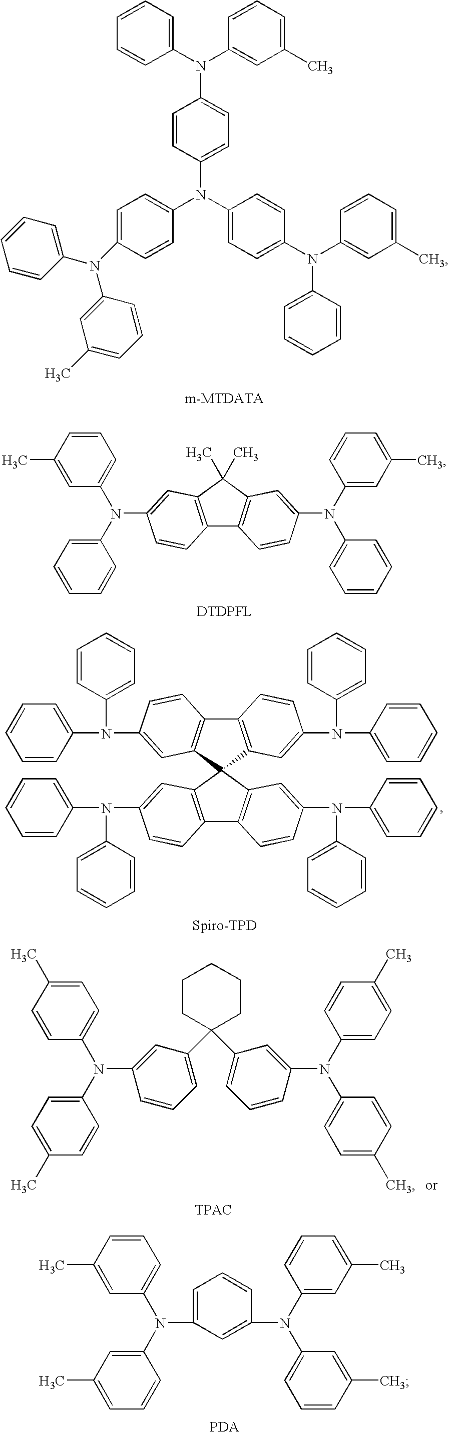

a stilbene based compound

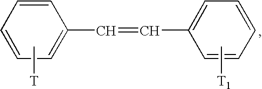

wherein T and T₁ stand for an organic radical;
a hydrazone based compound

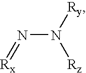

wherein Rₓ, R_{y} and R_{z} stand for an organic radical, and the like can be used.

Compounds to be used as a positive hole transporting material are not restricted to the above listed compounds. Any compound having a property of transporting positive holes can be used as a positive hole transporting material such as triazole derivatives, oxadiazole derivatives, imidazole derivatives, polyarylalkane derivatives, pyrazoline derivative, pyrazolone derivatives, phenylene diamine derivatives, arylamine derivatives, amino substituted chalcone derivatives, oxazole derivatives, stilbenzylanthracene derivatives, fluorenone derivatives, hydrazone derivatives, stilbene derivatives, copolymers of aniline derivatives, PEDOT (poly(3,4-ethylenedioxy-thiophene)) and the derivatives thereof, electro-conductive oligomers, particularly thiophene oligomers, porphyrin compounds, aromatic tertiary amine compounds, stilbenzyl amine compounds etc.

Particularly, aromatic tertiary amine compounds such as N,N′-diphenyl-N,N′-(1-naphtyl)-1,1′-diphenyl-4,4′-diamine (α-NPD), N,N,N′,N′-tetraphenyl-4,4′-diaminobiphenyl, N,N′-diphenyl-N,N′-bis(3-methylphenyl)-4,4′-diaminobiphenyl (TPD), 2,2′-bis(di-p-torylaminophenyl)propane, 1,1′-bis(4-di-torylaminophenyl)-4-phenylcyclohexane, bis(4-dimethylamino-2-methylphenyl)phenylmethane, bis(4-di-p-tolylaminophenyl)phenyl-methane, N,N′-diphenyl-N,N′-di(4-methoxyphenyl)-4,4′-diaminobiphenyl, N,N,N′,N′-tetraphenyl-4,4′-diaminodiphenylether, 4,4′-bis(diphenylamino)quaterphenyl, N,N,N-tri(p-tolyl)amine, 4-(di-p-tolylamino)-4′-[4-(di-p-tolylamino)stilyl]stilbene, 4-N,N-diphenylamino-(2 diphenylvinyl)benzene, 3-methoxy-4′-N,N-diphenylaminostilbene, N-phenylcarbazole etc. are used.

Furthermore, 4,4′-bis[N-(1-naphtyl)-N-phenylamino]biphenyl disclosed in U.S. Pat. No. 5,061,569 and the compounds disclosed in EP-A 508,562, in which three triphenylamine units are bound to a nitrogen atom, such as 4,4′,4″-tris[N-(3-methylphenyl)-N-phenylamino]triphenylamine, can be used.

A positive hole transporting layer can be formed by preparing an organic film containing at least one positive hole transporting material on the anode. The positive hole transporting layer can be formed by the vacuum deposition method, the spin-coating method, the casting method, ink jet printing method, the LB method and the like. Of these methods, the vacuum deposition method, the spin-coating method and the casting method are particularly preferred in view of ease and cost.

In the case of using the vacuum deposition method, the conditions for deposition may be chosen in the same manner as described for the formation of a light emitting layer (see above). If it is desired to form a positive hole transporting layer comprising more than one positive hole transporting material, the coevaporation method can be employed using the desired compounds.

In the case of forming a positive hole transporting layer by the spin-coating method or the casting method, the layer can be formed under the conditions described for the formation of the light emitting layer (see above).

As in the case of forming the light emitting layer a smoother and more homogeneous positive hole transporting layer can be formed by using a solution containing a binder and at least one positive hole transporting material. The coating using such a solution can be performed in the same manner as described for the light emitting layer. Any polymer binder may be used, provided that it is soluble in the solvent in which the at least one positive hole transporting material is dissolved. Examples of appropriate polymer binders and of appropriate and preferred concentrations are given above when describing the formation of a light emitting layer.

The thickness of the positive hole transporting layer is preferably chosen in the range of from 0.5 to 1000 nm, preferably from 1 to 100 nm, more preferably from 2 to 50 nm.

As hole injection materials known organic hole transporting compounds such as metal-free phthalocyanine (H₂Pc), copper-phthalocyanine (Cu—Pc) and their derivatives as described, for example, in JP64-7635 can be used. Furthermore, some of the aromatic amines defined as hole transporting materials above, which have a lower ionisation potential than the hole transporting layer, can be used.

A hole injection layer can be formed by preparing an organic film containing at least one hole injection material between the anode layer and the hole transporting layer. The hole injection layer can be formed by the vacuum deposition method, the spin-coating method, the casting method, the LB method and the like. The thickness of the layer is preferably from 5 nm to 5 μm, and more preferably from 10 nm to 100 nm.

The electron transporting materials should have a high electron injection efficiency (from the cathode) and a high electron mobility. The following materials can be exemplified for electron transporting materials: tris(8-hydroxyquinolinato)-aluminum(III) and its derivatives, 2,2′,2″-(1,3,5-phenylene)tris-[1-phenyl-1H-benzimidazole] and it's derivatives, bis(10-hydroxybenzo[h]quinolinolato)beryllium(II) and its derivatives, oxadiazole derivatives, such as 2-(4-biphenyl)-5-(4-tert.-butylphenyl)-1,3,4-oxadiazole and its dimer systems, such as 1,3-bis(4-tert.-butylphenyl-1,3,4)oxadiazolyl)biphenylene and 1,3-bis(4-tert.-butylphenyl-1,3,4-oxadiazolyl)phenylene, dioxazole derivatives, triazole derivatives, coumarine derivatives, imidazopyridine derivatives, phenanthroline derivatives or perylene tetracarboxylic acid derivatives disclosed in Appl. Phys. Lett. 48 (2) (1986) 183.

An electron transporting layer can be formed by preparing an organic film containing at least one electron transporting material on the hole transporting layer or on the light-emitting layer. The electron transporting layer can be formed by the vacuum deposition method, the spin-coating method, the casting method, the LB method and the like.

It is preferred that the hole blocking materials for a positive hole inhibiting layer have high electron injection/transporting efficiency from the electron transporting layer to the light emission layer and also have higher ionisation potential than the light emitting layer to prevent the flowing out of positive holes from the light emitting layer to avoid a drop in luminescence efficiency.

As a hole-blocking material known materials, such as BAlq, TAZ, GaqMe₂Cl, TPBI and phenanthroline derivatives, e.g. bathocuproine (BCP), can be used:

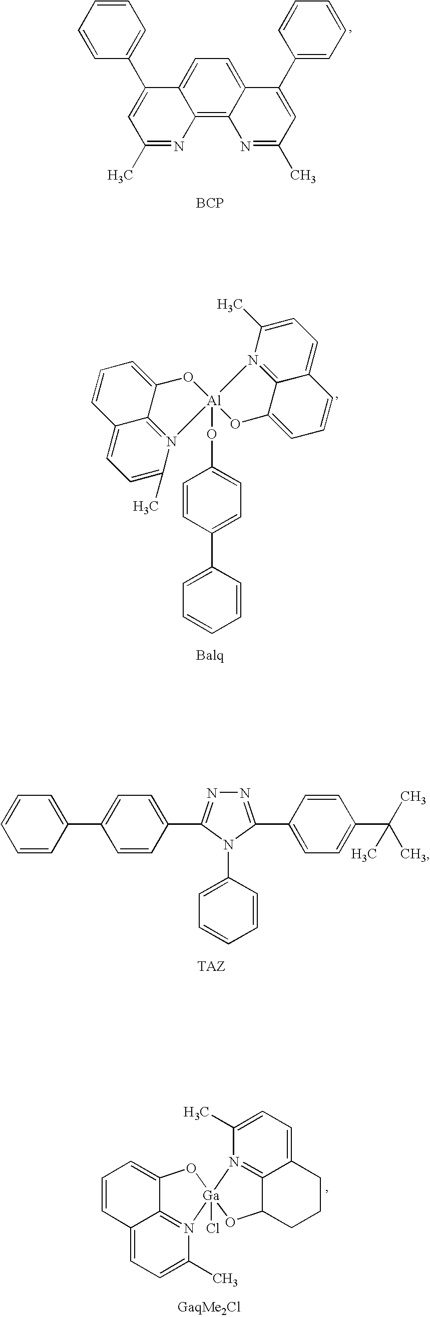

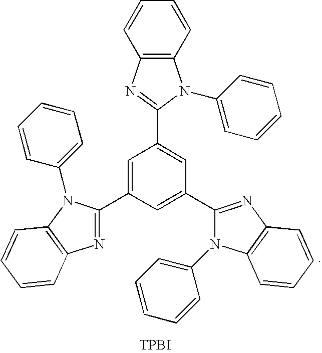

The hole-blocking layer can be formed by preparing an organic film containing at least one hole-blocking material between the electron transporting layer and the light-emitting layer. The hole-blocking layer can be formed by the vacuum deposition method, the spin-coating method, the casting method, ink jet printing method, the LB method and the like. The thickness of the layer preferably is chosen within the range of from 5 nm to 2 μm, and more preferably, within the range of from 10 nm to 100 nm.

As in the case of forming a light emitting layer or a positive hole transporting layer, a smoother and more homogeneous electron transporting layer can be formed by using a solution containing a binder and at least one electron transporting material.

The thickness of an electron transporting layer is preferably chosen in the range of from 0.5 to 1000 nm, preferably from 1 to 100 nm, more preferably from 2 to 50 nm.

The inventive compounds preferably exhibit an absorption maximum in the range of 330 to 500 nm.

The light-emitting compositions usually exhibit a fluorescence quantum yield (“FQY”) in the range of from 1>FQY≧0.1 (measured in aerated toluene or DMF). Further, in general, the inventive compositions exhibit a molar absorption coefficient in the range of from 1000 to 100000.

Another embodiment of the present invention relates to a method of coloring high molecular weight organic materials (having a molecular weight usually in the range of from 10³ to 10⁷ g/mol; comprising biopolymers, and plastic materials, including fibres) by incorporating therein the inventive compounds or compositions by methods known in the art.

The inventive compounds and compositions can be used, as described for DPP compounds in EP-A-1087005, for the preparation of
inks, for printing inks in printing processes, for flexographic printing, screen printing, packaging printing, security ink printing, intaglio printing or offset printing, for pre-press stages and for textile printing, for office, home applications or graphics applications, such as for paper goods, for example, for ballpoint pens, felt tips, fiber tips, card, wood, (wood) stains, metal, inking pads or inks for impact printing processes (with impact-pressure ink ribbons), for the preparation of
colorants, for coating materials, for industrial or commercial use, for textile decoration and industrial marking, for roller coatings or powder coatings or for automotive finishes, for high-solids (low-solvent), water-containing or metallic coating materials or for pigmented formulations for aqueous paints, for the preparation of
pigmented plastics for coatings, fibers, platters or mold carriers, for the preparation of
non-impact-printing material for digital printing, for the thermal wax transfer printing process, the ink jet printing process or for the thermal transfer printing process, and also for the preparation of
color filters, especially for visible light in the range from 400 to 700 nm, for liquid-crystal displays (LCDs) or charge combined devices (CCDs) or for the preparation of cosmetics or for the preparation of
polymeric ink particles, toners, dye lasers, dry copy toners liquid copy toners, or electrophotographic toners, and electroluminescent devices.

Another preferred embodiment concerns the use of the inventive compounds and compositions for color changing media. There are two major techniques in order to realize full-color organic electroluminescent devices:

(i) conversion of the electroluminescent blue or white to photoluminescent green and red via color changing media (CCM), which absorb the above electroluminescent blue, and fluorescence in green and red.
(ii) conversion of the white luminescent emission to blue, green and red via color filters.

The inventive compounds or compositions are useful for EL materials for the above category (i) and, in addition, for the above mention technique (ii). This is because the invented compounds or compositions can exhibit strong photoluminescence as well as electroluminescence

Technique (i) is, for example, known from U.S. Pat. No. 5,126,214, wherein EL blue with a maximum wavelength of ca. 470-480 nm is converted to green and red using coumarin, 4-(dicyanomethylene)-2-methyl-6-(p-dimethylaminostyryl)-4H-pyran, pyridine, rhodamine 6G, phenoxazone or other dyes.

Illustrative examples of suitable organic materials of high molecular weight which can be colored with the inventive compositions are described in EP-A-1087005.

Particularly preferred high molecular weight organic materials, in particular for the preparation of a paint system, a printing ink or ink, are, for example, cellulose ethers and esters, e.g. ethylcellulose, nitrocellulose, cellulose acetate and cellulose butyrate, natural resins or synthetic resins (polymerization or condensation resins) such as aminoplasts, in particular urea/formaldehyde and melamine/formaldehyde resins, alkyd resins, phenolic plastics, polycarbonates, polyolefins, polystyrene, polyvinyl chloride, polyamides, poly-urethanes, polyester, ABS, ASA, polyphenylene oxides, vulcanized rubber, casein, silicone and silicone resins as well as their possible mixtures with one another.

It is also possible to use high molecular weight organic materials in dissolved form as film formers, for example boiled linseed oil, nitrocellulose, alkyd resins, phenolic resins, melamine/formaldehyde and urea/formaldehyde resins as well as acrylic resins.

Said high molecular weight organic materials may be obtained singly or in admixture, for example in the form of granules, plastic materials, melts or in the form of solutions, in particular for the preparation of spinning solutions, paint systems, coating materials, inks or printing inks.

In a particularly preferred embodiment of this invention, the inventive compounds and corm positions are used for the mass coloration of polyvinyl chloride, polyamides and, especially, polyolefins such as polyethylene and polypropylene as well as for the preparation of paint systems, including powder coatings, inks, printing inks, color filters and coating colors.

Illustrative examples of preferred binders for paint systems are alkyd/melamine resin paints, acryl/melamine resin paints, cellulose acetate/cellulose butyrate paints and two-pack system lacquers based on acrylic resins which are crosslinkable with polyisocyanate.

Hence, another embodiment of the present invention relates to a composition comprising
(a) 0.01 to 50, preferably 0.01 to 5, particularly preferred 0.01 to 2% by weight, based on the total weight of the coloured high molecular organic material, of a fluorescent diketopyrrolopyrrole according to formula I or of a composition according to the present invention, and
(b) 99.99 to 50, preferably 99.99 to 95, particularly preferred 99.99 to 98% by weight, based on the total weight of the coloured high molecular organic material, of a high molecular organic material, and
(c) optionally, customary additives such as rheology improvers, dispersants, fillers, paint auxiliaries, siccatives, plasticizers, UV-stabilizers, and/or additional pigments or corresponding precursors in effective amounts, such as e.g. from 0 to 50% by weight, based on the total weight of (a) and (b).

To obtain different shades, the inventive fluorescent DPP compounds of formula I or the inventive compositions may advantageously be used in admixture with fillers, transparent and opaque white, colored and/or black pigments as well as customary luster pigments in the desired amount.

For the preparation of paints systems, coating materials, color filters, inks and printing inks, the corresponding high molecular weight organic materials, such as binders, synthetic resin dispersions etc. and the inventive compounds or compositions are usually dispersed or dissolved together, if desired together with customary additives such as dispersants, fillers, paint auxiliaries, siccatives, plasticizers and/or additional pigments or pigment precursors, in a common solvent or mixture of solvents. This can be achieved by dispersing or dissolving the individual components by themselves, or also several components together, and only then bringing all components together, or by adding everything together at once.

Hence, a further embodiment of the present invention relates to a method of using the inventive compounds or compositions for the preparation of dispersions and the corresponding dispersions, and paint systems, coating materials, inks and printing inks comprising the inventive compositions.

A particularly preferred embodiment relates to the use of the inventive compounds or compositions for the preparation of fluorescent tracers for e.g. leak detection of fluids such as lubricants, cooling systems etc., as well as to fluorescent tracers or lubricants comprising the inventive compositions.

For the pigmentation of high molecular weight organic material, the inventive compounds or compositions, optionally in the form of masterbatches, are mixed with the high molecular weight organic materials using roll mills, mixing apparatus or grinding apparatus. Generally, the pigmented material is subsequently brought into the desired final form by conventional processes, such as calandering, compression molding, extrusion, spreading, casting or injection molding.

For pigmenting lacquers, coating materials and printing inks the high molecular weight organic materials and the inventive compounds or compositions, alone or together with additives, such as fillers, other pigments, siccatives or plasticizers, are generally dissolved or dispersed in a common organic solvent or solvent mixture. In this case it is possible to adopt a procedure whereby the individual components are dispersed or dissolved individually or else two or more are dispersed or dissolved together and only then are all of the components combined.

The present invention additionally relates to inks comprising a coloristically effective amount of the pigment dispersion of the inventive compositions.

The weight ratio of the pigment dispersion to the ink in general is chosen in the range of from 0.001 to 75% by weight, preferably from 0.01 to 50% by weight, based on the overall weight of the ink.

The preparation and use of color filters or color-pigmented high molecular weight organic materials are well-known in the art and described e.g. in Displays 14/2, 1151 (1993), EP-A 784085, or GB-A 2,310,072.

The present invention relates, moreover, to toners comprising a pigment dispersion containing an inventive compound or composition or a high molecular weight organic material pigmented with an inventive composition in a coloristically effective amount.

The present invention additionally relates to colorants, colored plastics, polymeric ink particles, or non-impact-printing material comprising an inventive composition, preferably in the form of a dispersion, or a high molecular weight organic material pigmented with an inventive composition in a coloristically effective amount.

A coloristically effective amount of the pigment dispersion according to this invention comprising an inventive composition denotes in general from 0.0001 to 99.99% by weight, preferably from 0.001 to 50% by weight and, with particular preference, from 0.01 to 50% by weight, based on the overall weight of the material pigmented therewith.

The inventive compositions can be applied to colour polyamides, because they do not decompose during the incorporation into the polyamides. Further, they exhibit an exceptionally good lightfastness, a superior heat stability, especially in plastics.

The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light. The compounds and compositions of the present invention can be used in the fields of an organic EL device, an electrophotographic photoreceptor, a photoelectric converter, a solar cell, an image sensor, and the like.

The following examples are for illustrative purposes only and are not to be construed to limit the scope of the instant invention in any manner whatsoever. In the examples the “parts” denote “parts by weight” and the “percentages” denote “percentages by weight”, unless otherwise stated.

### EXAMPLE 1

### Synthesis of 4,7-Dihydroxy-1-oxo-3-phenyl-2-propyl-2,3-dihydro-1H-isoindole-5-carboxylic acid isopropyl ester

### 1a) 2-Benzoyl-succinic acid diethyl ester

To a solution of ethyl benzoacetate (5.07 g, 26.4 mmol) in dry acetone (15 ml) and DME (10 ml) is added ethyl bromoacetate (4.40 g, 26.4 mmol) and K₂CO₃ (3.64, 26.4 mmol) at room temperature, and the mixture is then stirred at reflux for 22 hours. After the reaction is completed, K₂CO₃ is removed by filtration, then the filtered solution is concentrated by evaporation. A brown oil crude product is obtained (7.25 g, 98.8%). The crude product is used in the next reaction step without further purification.

¹H-NMR spectrum (CDCl₃). δ [ppm]: 1.13-1.37 (m, 6H), 3.08 (m, 2H), 4.06-4.27 (m, 4H), 4.85 (t, 1H), 7.41-8.04 (m, 5H).

### 1b) 5-Oxo-2-phenyl-1-propyl-4,5-dihydro-1H-pyrrole-3-carboxylic acid ethyl ester

To acetic acid (25 ml) is added n-propylamine (10.63 g, 180.0 mmol) within 40 minutes at 0° C. The mixture is stirred under N₂ at room temperature for 40 minutes. 2-Benzoyl-succinic acid diethyl ester (5.00 g, 18.0 mmol) is then added to the above mixture within 5 minutes, and this is stirred at 100° C. overnight. The reaction mixture is then poured into ice-water (500 g), and the solution is extracted with ethyl acetate (3×150 ml), washed with water (2×200 ml), saturated NaHCO₃aq (300 ml), water (200 ml) and brine (200 ml), respectively. The obtained crude solution is dried over anhydrous MgSO₄, and then concentrated by evaporation. The brown crude product is purified by silica gel column chromatography with ethyl acetate-n-hexane solution (EA/H=1/5 solution). A pale brown solid product is obtained (3.84 g, 78.0%).

¹H-NMR spectrum (CDCl₃). δ [ppm]: 0.71 (t, 3H), 1.03 (t, 3H), 1.35 (dt, 2H), 3.29 (t, 2H), 3.46 (s, 2H), 3.99 (q, 2H), 7.31 (dd, 2H), 7.46 (m, 3H).

### 1c) 4,7-Dihydroxy-1-oxo-3-phenyl-2-propyl-2,3-dihydro-1H-isoindole-5-carboxylic acid isopropyl ester

To 2-methyl-2-butanol (80 ml) is added NaH (1.32 g, 33.0 mmol) for 10 minutes at room temperature. The mixture is stirred under N₂ at room temperature for 15 minutes to prepare sodium 2-methyl-2-butanoxide. The solution of 5-oxo-2-phenyl-1-propyl-4,5-dihydro-1H-pyrrole-3-carboxylic acid ethyl ester (3.00 g, 11.0 mmol) and diisopropyl succinate (5.50 g, 27.0 mmol) in 2-methyl-2-butanol is then added to the above mixture within 30 minutes, and this is stirred at 100° C. overnight. Sodium 2-methyl-2-butanoxide (22.0 mmol) is prepared by reacting NaH (0.88 g, 22.0 mmol) and 2-methyl-2-butanol (50 ml) and added to above reaction mixture and stirred at 110° C. overnight. The reaction mixture is then poured into water (300 ml), the solution is acidified to pH 3 with 1N HCl (50 ml), extracted with ethyl acetate (3×200 ml), washed with water (2×100 ml) and brine (200 ml), respectively. The obtained crude solution is dried over anhydrous MgSO₄ and then concentrated by evaporation. The brown crude product is purified by silica gel column chromatography with ethyl acetate-n-hexane solution (EA/H=1/20 to 1/3) twice. A beige solid product is obtained (50.0 mg, 1.2%; Mp: 125° C.). The structure is identified by MS structure analysis.

### EXAMPLE 2

### Synthesis of N-Benzyl-2,5-dihydroxy-terephthalamic acid ethyl ester

To a solution of 2,5-dihydroxy-terephthalic acid diethyl ester (1.0 g, 3.9 mmol) in dry DMF (15 ml) is added benzylamine (0.42 g, 3.9 mmol) by portion at 100° C., and the mixture is then stirred at 110° C. for 22 hours. After the reaction is completed, the reaction mixture is poured into H₂O (50 ml), then the solution is neutralized with 1N HCl (0.5 ml). The organic layer is washed with H₂O (50 ml) and saturated NaClaq (50 ml). Then the organic layer is dried over MgSO₄ and concentrated by evaporation. A yellow solid crude product is obtained. The crude product is purified by silica gel column chromatography with ethyl acetate-n-hexane solution (EA/H=1/5 to 1/3). A beige solid product is obtained. (420 mg, 33%). Mp: 110° C.; ¹H-NMR spectrum (CDCl₃). δ [ppm]: 1.39 (t, 3H), 4.39 (q, 2H), 4.60 (d, 2H), 6.60 (s, 1H), 6.91 (s, 1H), 7.32 (m, 5H), 7.46 (s, 1H), 10.1 (s, 1H), 11.4 (s, 1H).

### EXAMPLES 3-13

The compounds of examples 2 to 13 are prepared according to the method described in example 1 or 2 from the corresponding carboxylic acid, ester or amide. The compounds are listed in tables 1 and 2.

TABLE I

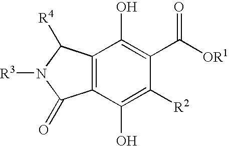

| Exam- | | | | | Melting point |
|---|---|---|---|---|---|
| ple | R¹ | R² | R³ | R⁴ | [C° C.] |
| 3 | isopropyl | H | n-propyl | methyl | 111 |
| 4 | isopropyl | H | H | 1-naphthyl | 275 |
| 5 | isopropyl | H | H | 9-phenanthrenyl | 302 |
| 6 | isopropyl | H | H | phenyl | 238 |
| 7 | isopropyl | H | H | methyl | 164 |
| 8 | isopropyl | H | H | 4-bromo-phenyl | |
| 9 | isopropyl | H | H | 4-methyl-phenyl | 288 |
| 13 | isopropyl | H | H | 2-methoxy-phenyl | 219 |

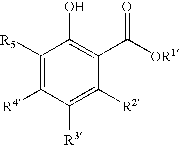

| | | | | | | Melting point |
|---|---|---|---|---|---|---|
| Example | R^{1′} | R^{2′} | R^{3′} | R^{4′} | R⁵ | (° C.) |
| 2 | ethyl | H | —OH | —C(O)NHCH₂Ph | H | 144 |
| 10 | ethyl | H | —OH | —C(O)NH-n-hexyl | H | 110 |
| 11 | ethyl | H | —OH | —C(O)morpholino | H | 199 |
| 12 | methyl | —OPh | —OH | —C(O)NH-n-hexyl | —OPh | 128 |

### EXAMPLE 14

A glass substrate (manufactured by Geomatek Co., a product prepared by electron beam vapor deposition method) on which an ITO transparent electroconductive film had been deposited up to a thickness of ca. 150 nm is cut into a size of 10×20 mm and etched. The substrate thus obtained is subjected to ultrasonic washing with detergent water for 15 minutes, and then washing with pure water. Subsequently, the substrate is subjected to ultrasonic washing with acetone for 15 minutes, and then dried. Just before forming the substrate into an element, the substrate is subjected to a plasma treatment for half hour and placed in a vacuum vapour deposition apparatus, and the apparatus is evacuated until the inner pressure reached 1×10⁻⁵ Pa or less. Then, according to the resistance heating method, firstly CuPc (20 nm) and N,N′-diphenyl-N,N′-(1-naphtyl)-1,1′-diphenyl-4,4′-diamine (α-NPD) are vapor-deposited successively as a positive hole transporting material up to a thickness of 40 nm, to form a positive hole transporting layer. Subsequently, 4,4′-N,N′-dicarbazole-biphenyl (CBP) and the compound of example 1 are co-deposited as a light emitting layer up to a thickness of 30 nm by controlling the ratio of deposition rate (CBP: compound of Example 3=99: ca. 1) to form an uniform light emitting layer. Subsequently, a bathocuproine (BCP) layer is vapor-deposited to form a hole blocking layer having a thickness of 10 nm. Subsequently, a Alq₃ layer is vapor-deposited to form an electron transporting layer having a thickness of 20 nm. Subsequently, a LiF layer is vapor-deposited to form an electron injection layer having a thickness of 0.5 nm, On top of that, Al is vapor-deposited to form a cathode having a thickness of 100 nm, whereby an element having a size of 2×2 mm square is prepared.

When a voltage of 13 V is applied to the device, 800 cd/m² of white luminescent was observed. The EL spectrum is described in FIG. 1. The color point is x=0.25, y=0.32.

### EXAMPLE 15

An electroluminescent device is prepared in the same manner as in Example 15, except that the compound of Example 5 is used without CBP as light emitting layer and BCP (hole blocking layer) is not used. When a voltage of 18 V is applied to the device, 40 cd/m² of white luminescent is observed.

1. A compound of the formula: 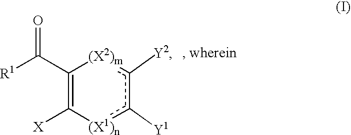 [symbol]
is a single or double bond, m and n are independently of each other 0 or 1,
when n is 0 and m is 1, 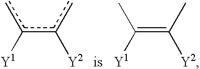
X² is —C(R²)═C(R³)—, —C(R⁴)(R⁵)—C(R⁴)(R⁵)—, —C(R²)═C(R³)—C(R⁴)(R⁵)—C(R⁴)(R⁵)—C(R²)═C(R³)—, —C(R⁴)(R⁵)—C(R⁴)(R⁵)—C(R²)═C(R³)—, —C(R²)═C(R³)—C(R⁴)(R⁵)—C(R⁴)(R⁵)—, or —C(R²)═C(R³)—C(R²)═C(R³)—;
when m is 0 and n is 1, 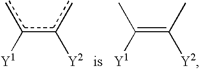
X¹ is —C(R²)═C(R³)—, —C(R⁴)(R⁵)—C(R⁴)(R⁵)—, —C(R²)═C(R³)—C(R⁴)(R⁵)—, —C(R⁴)(R⁵)—C(R²)═C(R³)—, —C(R⁴)(R⁵)—C(R⁴)(R⁵)—C(R²)═C(R³)—, —C(R²)═C(R³)—C(R⁴)(R⁵)—C(R⁴)(R⁵)—, or —C(R²)═C(R³)—C(R²)═C(R³)—;
when m is 1 and n is 1, 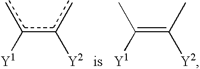
X¹ and X² are independently of each other —C(R⁴)(R⁵)—, —C(R⁴)(R⁵)—C(R⁴)(R⁵)—, or —C(R²)═C(R³)—; or
when m is 1 and n is 1, 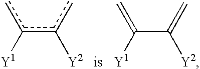
X¹ and X² are independently of each other —C(R²)—, —C(R³)—, —C(R³)—C(R⁴)(R⁵)—, or —C(R²)—C(R⁴)(R⁵)—;
Y¹ is —OH, Y² is —CO—NR⁸R⁹, or Y¹¹; or
Y² is —OH, Y¹ is —CO—NR⁸R⁹, or Y¹¹; wherein
Y¹¹ is 2H-2- or 5-pyrrolyl, imidazolyl, 3- or 5-pyrazolyl, 2- or 4-thiazolyl, 2- or 4-oxazolyl, 3-isoxazolyl, 2- or 6-pyridyl, pyrazinyl, 3- or 6-pyridazinyl, triazinyl, 2-benzimidazolyl, 2-benzothiazolyl, 2-benzoxazolyl, 3- or 4-benzothiadiazolyl, 1-triazolyl, 3-indazolyl, 2-quinolyl, 1- or 3-isoquinolyl, 1- or 4-phthalazinyl, 2- or 3-quinoxalinyl, pteridinyl, each of which can optionally be substituted 1 to 7 times by halogen, C₁-C₁₈alkyl, C₃-C₈cycloalkyl, benzyl and/or phenoxycarbonyl; phenyl which can optionally be substituted by one or more —OR⁶, —SR⁷ and/or —NR⁸R⁹; C₂-C₁₂alkoxycarbonyl, optionally interrupted by one or more —O—, —S— and/or substituted by one or more hydroxyl groups; —OR⁶, —SR⁷, —SOR⁷, —SO₂R⁷ and/or —NR⁸R⁹; C₁-C₈alkanoyl, or benzoyl which can optionally be substituted by —OR⁶, —SR⁷, —SOR⁷, —SO₂R⁷, —NR⁸R⁹, morpholino and/or dimethylmorpholino; wherein R⁶, R⁷, R⁸ and/or R⁹ can optionally form 5-, 6- or 7-membered rings with further substituents on the heteroaromatic ring;
X is —OH, or —NR⁸R⁹,
R¹ is —OH, C₃-C₈cycloalkoxy, C₁-C₁₈alkoxy, C₃-C₆alkenoxy, or C₁-C₈thioalkoxy which can optionally be substituted by one or more groups halogen, —OR⁶, —SR⁷ and/or —CN; C₆-C₂₄aryloxy, C₇-C₂₄aralkyloxy, C₆-C₂₄thioaryloxy C₇-C₂₄thioaralkyloxy, which can optionally be substituted by one or more groups C₁-C₈alkyl, halogen, —CN, —OR⁶, —SR⁷ and/or —NR⁸R⁹;
R² and R³ are independently of each other hydrogen, 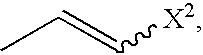
wherein X² is aryl, or heteroaryl, which can be substituted one to three times with C₁-C₈alkyl and/or C₁-C₈alkoxy; —OR⁶, —SR⁷ or —NR⁸R⁹; C₁-C₂₅alkyl which can optionally be substituted by one or more groups halogen, —OR⁶, phenyl, naphthyl and/or phenanthryl which can optionally be substituted by —OR⁶, —SR⁷ and/or —NR⁸R⁹; C₃-C₈cycloalkyl; C₂-C₂₀alkyl interrupted by one or more —O—, —S— and/or optionally substituted by one or more halogen, —OR⁶, phenyl which can optionally be substituted by —OR⁶, —SR⁷ and/or —NR⁸R⁹; aryl, or heteroaryl, such as Y¹¹, which can optionally be substituted by one or more groups C₁-C₈alkyl, phenyl, halogen, —OR⁶, —SR⁷ and/or —NR⁸R⁹; C₂-C₂₀alkanoyl, or benzoyl which can optionally be substituted by one or more groups C₁-C₈alkyl, phenyl, —OR⁶, —SR⁷ and/or —NR⁸R⁹; C₂-C₁₂alkoxycarbonyl optionally interrupted by one or more —O—, —S— and/or optionally substituted by one or more hydroxyl groups; phenoxycarbonyl which can optionally be substituted by C₁-C₈alkyl, halogen, phenyl, —OR⁶, —SR⁷ and/or —NR⁸R⁹; —CN, COOH, —CO—NR⁸R⁹, —NO₂, C₁-C₄haloalkyl, —S(O)₁₋₂—C₁-C₈alkyl or —S(O)₁₋₂-phenyl which can optionally be substituted by C₁-C₁₂alkyl; —SO₂O-phenyl which can optionally be substituted by C₁-C₁₂alkyl; wherein the substituents R¹, R², R³, Y and Y¹ can optionally form 5-, 6- or 7-membered rings with each other;
R⁴ and R⁵ are as defined for R², or R⁴ and R⁵ form 5-, 6- or 7-membered rings with each other, which may be substituted,
R⁶ is hydrogen, C₁-C₂₀alkyl, phenyl-C₁-C₃alkyl; C₁-C₈alkyl which is substituted by —OH, —SH, —CN, C₃-C₆alkenoxy, —OCH₂CH₂CN, —OCH₂CH₂(CO)O(C₁-C₄alkyl), —O(CO)—(C₁-C₄alkyl), —O(CO)-phenyl, —(CO)OH and/or —(CO)O(C₁-C₄alkyl); C₂-C₁₂alkyl which is interrupted by one or more —O—, —S—; —(CH₂CH₂O)ₙH, (CH₂CH₂O)ₙ(CO)—(C₁-C₈alkyl), C₁-C₈alkanoyl, C₂-C₁₂alkenyl, C₃-C₆alkenoyl, C₃-C₈cycloalkyl; benzoyl which can optionally be substituted by one or more groups C₁-C₈alkyl, halogen, —OH and/or C₁-C₄alkoxy; phenyl or naphthyl each of which can optionally be substituted by halogen, —OH, C₁-C₁₂alkyl, C₁-C₁₂-alkoxy, phenoxy, C₁-C₁₂alkylsulfanilyl, phenylsulfanilyl, —N(C₁-C₁₂alkyl)₂ and/or diphenylamino;
R⁷ is hydrogen, C₁-C₂₀alkyl, C₂-C₁₂alkenyl, C₃-C₈cycloalkyl, phenyl-C₁-C₃alkyl; C₁-C₈alkyl which is substituted by —OH, —SH, —CN, C₃-C₆alkenoxy, —OCH₂CH₂CN, —OCH₂CH₂(CO)O(C₁-C₄alkyl), —O(CO)—(C₁-C₄alkyl), —O(CO)-phenyl, —(CO)OH or —(CO)O(C₁-C₄alkyl); C₂-C₁₂alkyl which is interrupted by one or more —O—, or —S—; —(CH₂CH₂O)ₜH, —(CH₂CH₂O)ₜ(CO)—(C₁-C₈alkyl), C₁-C₈alkanoyl, C₂-C₁₂alkenyl, C₃-C₆alkenoyl; benzoyl which can optionally be substituted by one or more groups C₁-C₈alkyl, halogen, —OH, C₁-C₄alkoxy, or C₁-C₄alkylsulfanilyl; phenyl or naphthyl, each of which can optionally be substituted by halogen, C₁-C₁₂alkyl, C₁-C₁₂alkoxy, phenyl-C₁-C₃alkyloxy, phenoxy, C₁-C₁₂alkylsulfanilyl, phenylsulfanilyl, —N(C₁-C₁₂alkyl)₂, diphenylamino, —(CO)O(C₁-C₈alkyl), —(CO)—C₁-C₈alkyl, or —(CO)N(C₁-C₈alkyl)₂;
t is 1 to 20;
R⁸ and R⁹ are independently of each other hydrogen, C₁-C₂₀alkyl, C₂-C₄-hydroxyalkyl, C₂-C₁₀-alkoxyalkyl, C₂-C₅alkenyl, C₃-C₈cycloalkyl, C₇-C₂₄aralkyl, C₁-C₈alkanoyl, C₃-C₁₂-alkenoyl, formyl, benzoyl; C₆-C₂₄aryl, which can optionally be substituted by C₁-C₁₂alkyl, benzoyl, or C₁-C₁₂alkoxy; or R⁸ and R⁹ together are C₂-C₈alkylene optionally interrupted by —O—, —S— or —NR¹¹³— and/or optionally substituted by hydroxyl, C₁-C₄alkoxy, C₂-C₄alkanoyloxy, benzoyloxy, C₁-C₁₂alkylsulfanilyl, or phenylsulfanilyl which can optionally be substituted by one or more C₁-C₈alkyl, halogen, —OH and/or C₁-C₄alkoxy; or R⁸ and/or R⁹ together with a substituent, which is adjacent to the group —CO—NR⁸R⁹, or —NR⁸R⁹, form 5-, 6- or 7-membered rings, which may be substituted; R¹⁶³ is a hydrogen atom, a C₁-C₂₅alkyl group, which might be interrupted by —O—, a cycloalkyl group, an aralkyl group, an aryl group, or a heterocyclic group.
with the proviso that the following compounds are excluded:

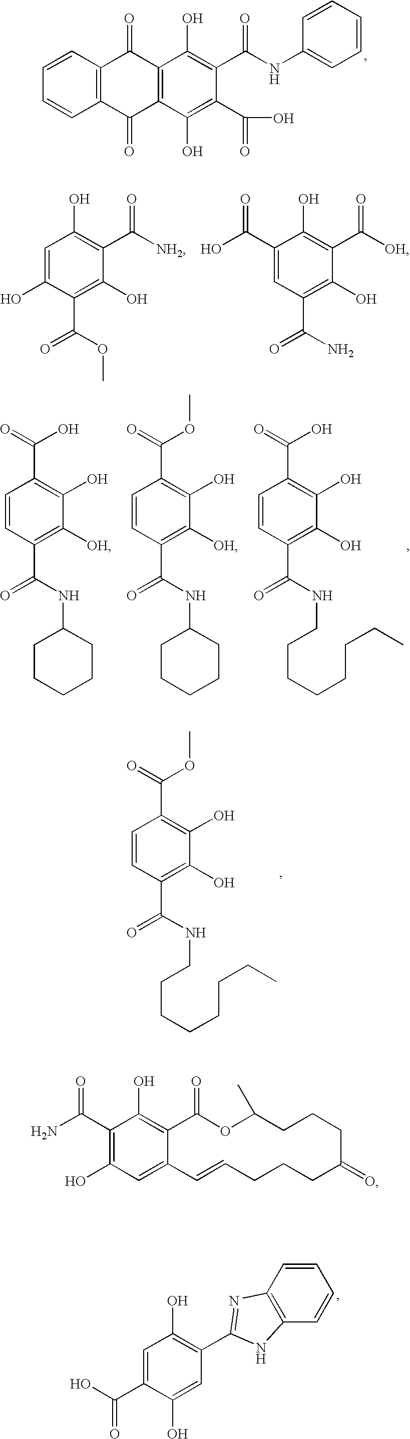

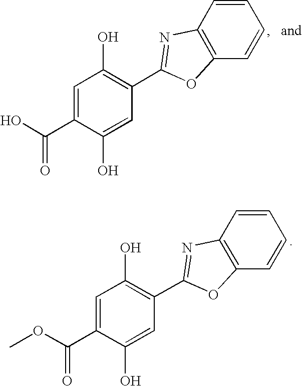

2. A compound according to claim 1 of the formula 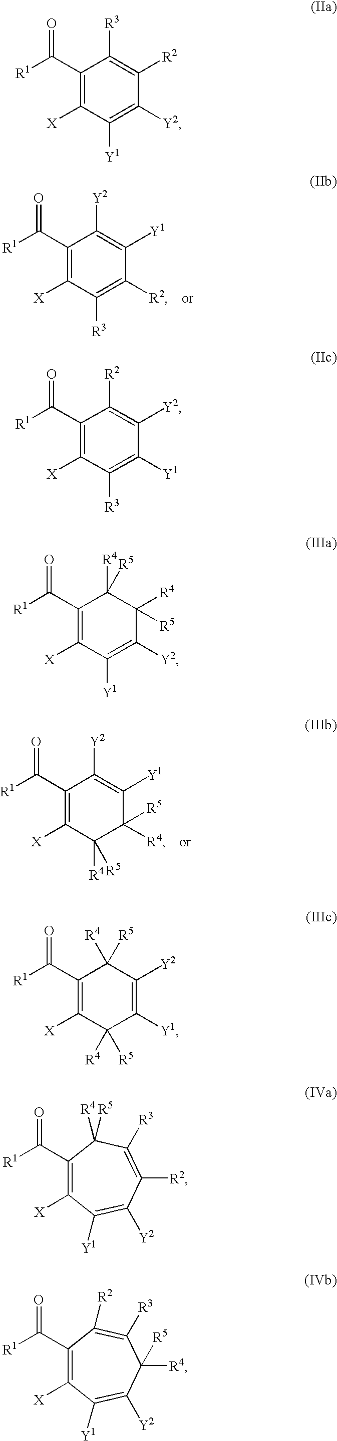 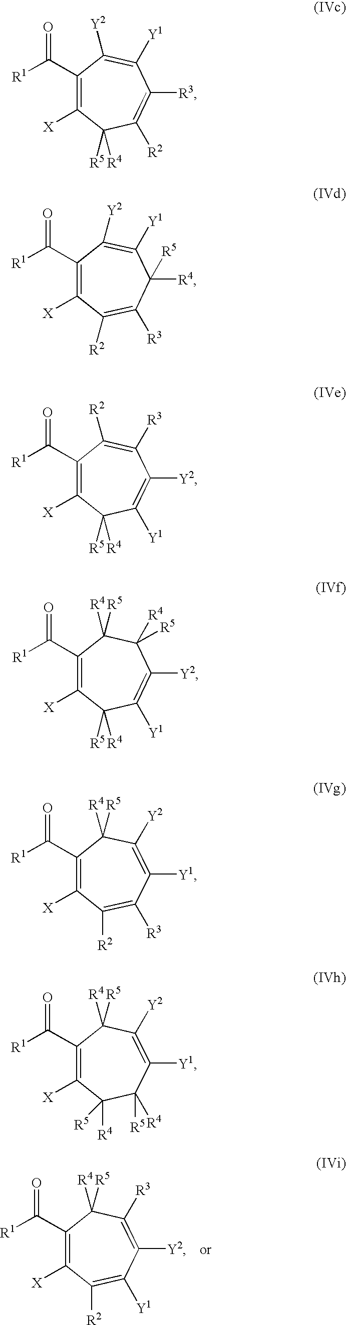 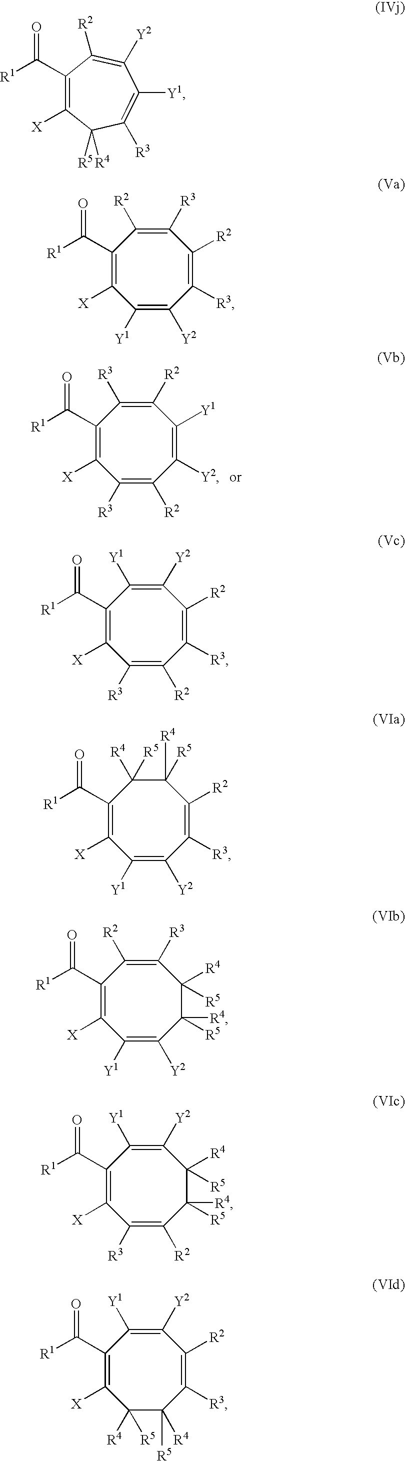 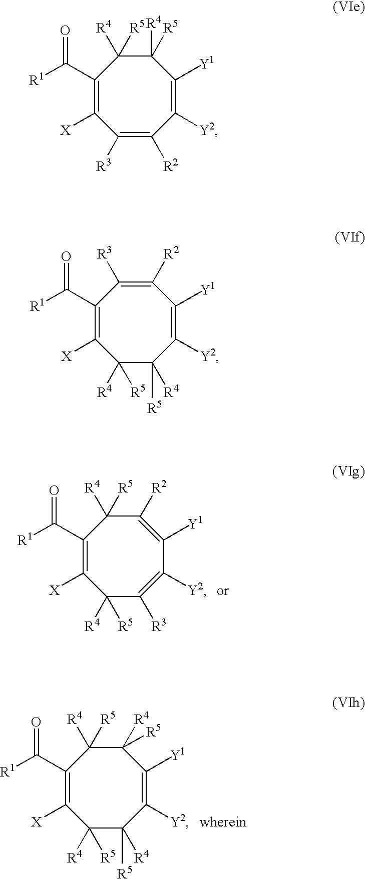
Y¹ is —OH, Y² is —CO—NR⁸R⁹, or Y¹¹; or
Y² is —OH, Y¹ is —CO—NR⁸R⁹, or Y¹¹; wherein
Y¹¹ is 2H-2- or 5-pyrrolyl, imidazolyl, 3- or 5-pyrazolyl, 2- or 4-thiazolyl, 2- or 4-oxazolyl, 3-isoxazolyl, 2- or 6-pyridyl, pyrazinyl, 3- or 6-pyridazinyl, triazinyl, 2-benzimidazolyl, 2-benzothiazolyl, 2-benzoxazolyl, 3- or 4-benzothiadiazolyl, 1-triazolyl, each of which can optionally be substituted 1 to 4 times by halogen, C₁-C₁₈alkyl, benzyl and/or phenoxycarbonyl; phenyl which can optionally be substituted by one or more —OR⁶, —SR⁷ and/or —NR⁸R⁹; C₂-C₁₂alkoxycarbonyl, optionally interrupted by one or more —O—, —S— and/or substituted by one or more hydroxyl groups; —OR⁶ and/or —NR⁸R⁹; wherein R⁶, R⁷, R⁸ and/or R⁹ can optionally form 5-, 6- or 7-membered rings with further substituents on the heteroaromatic ring;
X is —OH, or —NR⁸R⁹,
R¹ is —OH, C₃-C₈cycloalkoxy, C₁-C₁₈alkoxy, C₃-C₆alkenoxy, or C₁-C₈thioalkoxy which can optionally be substituted by one or more groups halogen, —OR⁶, —SR⁷ and/or —CN; C₆-C₂₄aryloxy, C₇-C₂₄aralkyloxy, C₆-C₂₄thioaryloxy, or C₇-C₂₄thioaralkyloxy, which can optionally be substituted by one or more groups C₁-C₈alkyl, halogen, —CN, —OR⁶, —SR⁷ and/or —NR⁸R⁹;
R² and R³ are independently of each other hydrogen, 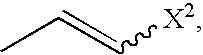
wherein X² is aryl, or heteroaryl, which can be substituted one to three times with C₁-C₈alkyl and/or C₁-C₈alkoxy; —OR⁶, —SR⁷ or —NR⁸R⁹; C₁-C₁₈alkyl which can optionally be substituted by one or more groups halogen, —OR⁶, phenyl, naphthyl and/or phenanthryl which can optionally be substituted by —OR⁶, —SR⁷ and/or —NR⁸R⁹; C₃-C₈cycloalkyl; C₂-C₁₂alkyl interrupted by one or more —O—, —S— and/or optionally substituted by one or more halogen, —OR⁶, phenyl which can optionally be substituted by —OR⁶, —SR⁷ and/or —NR⁸R⁹; aryl or heteroaryl which can optionally be substituted by one or more groups C₁-C₈alkyl, phenyl, halogen, —OR⁶, —SR⁷ and/or —NR⁸R⁹; C₂-C₁₂alkanoyl; C₂-C₁₂alkoxycarbonyl optionally interrupted by one or more —O—, —S— and/or optionally substituted by one or more hydroxyl groups; phenoxycarbonyl which can optionally be substituted by C₁-C₈alkyl, halogen, phenyl, —OR⁶, —SR⁷ and/or —NR⁸R⁹, —CO—NR⁸R⁹, C₁-C₄haloalkyl; wherein the substituents R¹ and R², or R³; R² and R³; R⁴ and R⁵; R¹, R², R³, R⁴, or R⁵ and X, Y¹, or Y² can optionally form 5-, 6- or 7-membered rings with each other, which can optionally be substituted;
R⁴ and R⁵ are as defined for R²,
R⁶ is hydrogen, C₁-C₁₂alkyl, phenyl-C₁-C₃alkyl; C₁-C₈alkyl which is substituted by —OH, —SH, —CN; C₂-C₁₂alkyl which is interrupted by one or more —O—, —S—; C₁-C₈alkanoyl; phenyl or naphthyl each of which can optionally be substituted by halogen, —OH, C₁-C₁₂alkyl, C₁-C₁₂-alkoxy, phenoxy, C₁-C₁₂alkylsulfanilyl, phenylsulfanilyl, —N(C₁-C₁₂alkyl)₂ and/or diphenylamino;
R⁷ is hydrogen, C₁-C₁₂alkyl; C₁-C₈alkyl which is substituted by —OH, —SH, —CN; C₂-C₁₂alkyl which is interrupted by one or more —O—, or —S—; phenyl or naphthyl, each of which can optionally be substituted by halogen, C₁-C₁₂alkyl, C₁-C₁₂alkoxy, phenyl-C₁-C₃alkyloxy, phenoxy, C₁-C₁₂alkylsulfanilyl, phenylsulfanilyl, —N(C₁-C₁₂alkyl)₂, diphenylamino;
R⁸ and R⁹ are independently of each other hydrogen, C₁-C₂₅alkyl, C₂-C₄-hydroxyalkyl, C₂-C₁₀-alkoxyalkyl, C₁-C₈alkanoyl, formyl, benzoyl; C₇-C₂₄aralkyl, especially phenyl-C₁-C₃alkyl, C₆-C₂₄aryl, especially phenyl, or naphthyl, each of which can optionally be substituted by C₁-C₁₂alkyl, benzoyl, or C₁-C₁₂alkoxy; or R⁸ and R⁹ together are C₂-C₈alkylene optionally interrupted by —O—, —S— or —NR¹⁶³— and/or optionally substituted by hydroxyl, C₁-C₄alkoxy; or R⁸ and/or R⁹ together with a substituent, which is adjacent to the group —CO—NR⁸R⁹, or —NR⁸R⁹, form 5-, 6- or 7-membered rings, which may be substituted; R¹⁶³ is a hydrogen atom, a C₁-C₂₅alkyl group, which is optionally interrupted by —O—, a cycloalkyl group, an aralkyl group, an aryl group, or a heterocyclic group.

3. A compound according to claim 1 of formula 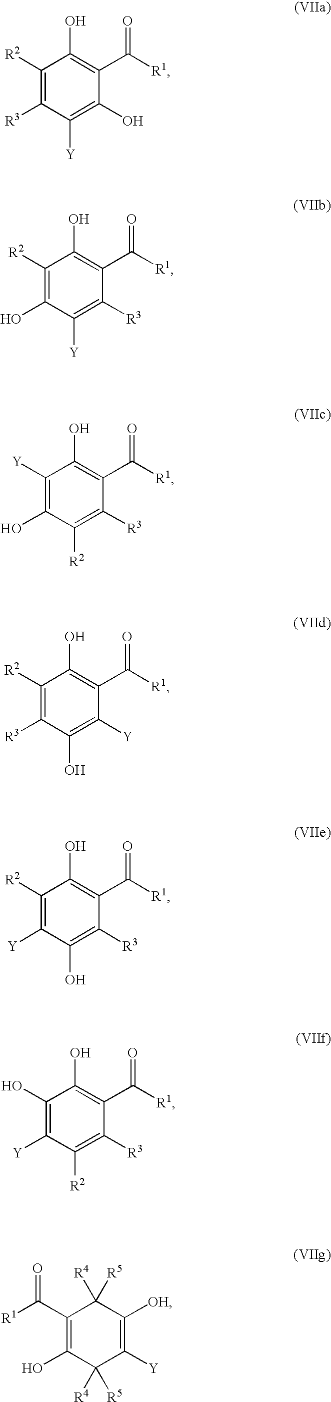 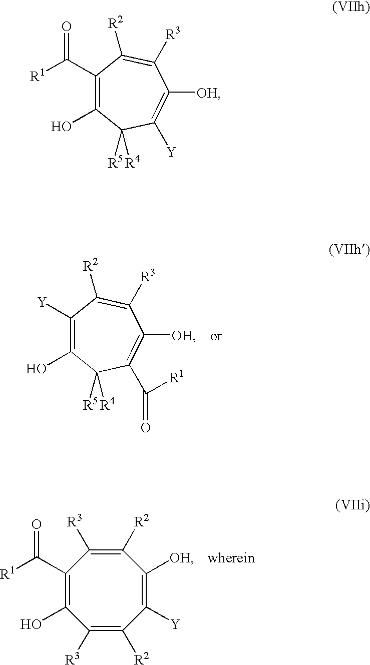
Y is —CO—NR⁸R⁹ imidazolyl, oxazolyl, thiazolyl, benzimidazolyl, benzoxazolyl or benzothiazolyl;
R¹ is —OH, C₆-C₂₄aryloxy, which can be substituted one to three times with C₁-C₈-alkyl, or C₁-C₈alkoxy; C₇-C₃₀aralkyloxy, which can be substituted one to three times with C₁-C₈-alkyl, or C₁-C₈alkoxy; C₁-C₁₈alkoxy which optionally forms 5- or 6-membered rings with adjacent substituents;
R² and R³ are independently of each other hydrogen, C₁-C₁₈alkoxy, C₁-C₁₈alkyl, 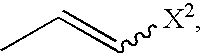
wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, C₆-C₂₄aryloxy, especially phenoxy, which can be substituted one to three times with C₁-C₈alkyl, C₇-C₂₄aralkyloxy, which can be substituted one to three times with C₁-C₈alkyl, C₇-C₂₄aralkyl, which can be substituted one to three times with C₁-C₈alkyl; wherein R² and R³ optionally form 5-, 6- or 7-membered rings with substituents R², R³ or Y;
R⁴ and R⁵ are independently of each other hydrogen, C₁-C₂₅alkyl, or R⁴ and R⁵ together form a C₅-C₇cycloalkyl ring, which can be substituted one to three times with C₁-C₈alkyl;
R⁸ and R⁹ are independently of each other hydrogen, C₁-C₂₅alkyl, or C₆-C₂₄aryl, which can be substituted one to three times with C₁-C₈alkyl, C₇-C₂₄aralkyl, which can be substituted one to three times with C₁-C₈alkyl; or R⁸ and R⁹ together are C₂-C₈alkylene optionally substituted by C₁-C₈alkyl and/or optionally interrupted by —O—, —S— or —NR¹⁶³; or if Y is —CO—NR⁸R⁹, R⁸ and R² or R³ form a five membered ring 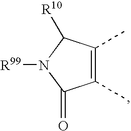
wherein R⁹⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³, wherein
R³³ and R³⁴ independently from each other stand for hydrogen or C₁-C₄alkyl, or phenyl, which can be substituted one to three times with C₁-C₄alkyl,
A³ stands for aryl or heteroaryl, which can be substituted one to three times with C₁-C₈alkyl and/or C₁-C₈alkoxy, and s stands for 0, 1, 2, 3 or 4,
R⁹ is as defined above and R¹⁰ is C₁-C₁₈alkyl; heteroaryl, or phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, halogen, or —OR⁶;
R⁶ is C₁-C₈alkyl; and
R¹⁶³ is a hydrogen atom, a C₁-C₂₅alkyl group, which is optionally interrupted by —O—, a cycloalkyl group, an aralkyl group, an aryl group, or a heterocyclic group.

4. A compound according to claim 1 of the formula 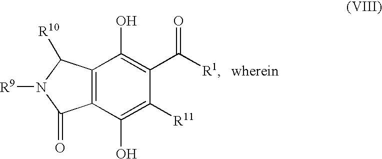
R¹ is —OH, C₇-C₃₀aralkyloxy, which can be substituted one to three times with C₁-C₈-alkyl, or C₁-C₈-alkoxy, C₆-C₂₄aryloxy, which can be substituted one to three times with C₁-C₈-alkyl, or C₁-C₈alkoxy,
R⁹ is selected from H, a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³, or —CR³³R³⁴—(CH₂)ₛ-A³ wherein
R³³ and R³⁴ independently from each other stand for hydrogen or C₁-C₄alkyl, or phenyl, which can be substituted one to three times with C₁-C₄alkyl,
A³ stands for aryl or heteroaryl which can be substituted one to three times with C₁-C₈alkyl and/or C₁-C₈alkoxy, and s stands for 0, 1, 2, 3 or 4,
R¹⁰ stands for aryl or heteroaryl, which can be substituted one to three times with C₁-C₈alkyl and/or C₁-C₈alkoxy,
R¹¹ is H, C₁-C₈alkyl, C₆-C₁₄aryl, which can be optionally substituted by one or more C₁-C₈alkyl groups, or
R¹¹ forms 5-, 6- or 7-membered rings with R¹, which can optionally be substituted by aryl, or heteroaryl groups, especially phenyl, biphenyl, naphthyl, fluorenyl, anthryl, pyrenyl, phenanthryl, pyridyl, furanyl, benzofuranyl, triazinyl, or dibenzofuranyl, which can be substituted one to three times with C₁-C₈-alkyl; or which can be condensed one time by phenyl which can be substituted one to three times with C₁-C₄-alkyl.

5. A compound according to claim 1 of formula VIII 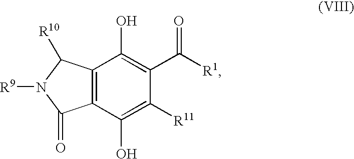
wherein
R¹⁰ is H, C₁-C₈alkyl; 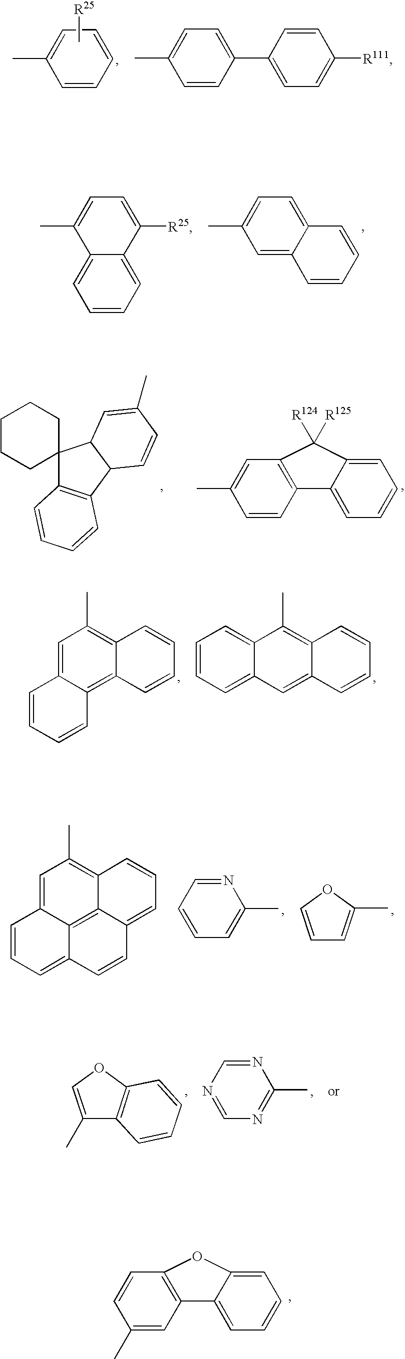
wherein R²⁵ and R¹¹¹ are independently of each other C₁-C₈-alkyl, phenyl, 1- or 2-naphthyl, and R¹²⁴ and R¹²⁵ may be the same or different and are C₁-C₁₈alkyl, which can optionally be interrupted by —O—;
R¹ is —OH, benzyloxy, which can be substituted one to three times with C₁-C₈-alkyl, phenoxy, which can be substituted one to three times with C₁-C₈-alkyl, or C₁-C₁₈alkoxy,
R⁹ is C₁-C₁₈alkyl, an aryl group, especially phenyl, 1- or 2-naphthyl, which can be substituted by one or more groups C₁-C₈alkyl, or C₅-C₈cycloalkyl, which can be substituted by one or more groups C₁-C₈alkyl,
R¹¹ is H, C₁-C₈alkyl, C₆-C₁₄aryl, which can be optionally substituted by one or more C₁-C₈alkyl groups, or
R¹¹ and R¹ together form a ring and are a group —CHR¹⁰⁰—O—, wherein R¹⁰⁰ is hydrogen, C₁-C₁₈alkyl, phenyl, biphenyl, naphthyl, fluorenyl, or phenanthryl, which can be substituted one to three times with C₁-C₈-alkyl, or
R¹¹ and R¹ together form a ring and are a group —CHR¹⁰¹CHR¹⁰²—O—, wherein R¹⁰¹ and R¹⁰² are independently of each other hydrogen, C₁-C₁₈alkyl, phenyl, biphenyl, naphthyl, fluorenyl, or phenanthryl, which can be substituted one to three times with C₁-C₈-alkyl, or
R¹¹ and R¹ together form a ring and are a group —CR¹⁰³═CR¹⁰⁴—O—, wherein R¹⁰³ and R¹⁰⁴ are independently of each other hydrogen, C₁-C₁₈alkyl, phenyl, biphenyl, naphthyl, fluorenyl, or phenanthryl, which can be substituted one to three times with C₁-C₈-alkyl, or
R¹⁰³ and R¹⁰⁴ together form a ring and are a group 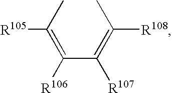
wherein R¹⁰⁵, R¹⁰⁶, R¹⁰⁸ and R¹⁰⁹ are independently of each other hydrogen, or C₁-C₁₈alkyl.

6. A compound according to claim 3 of the formula 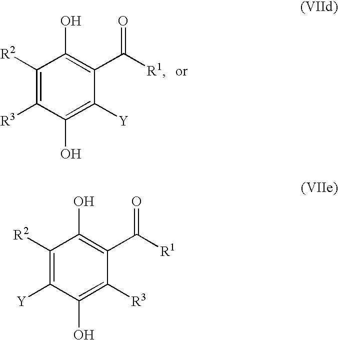
, wherein
R¹ is —OH, C₇-C₃₀aralkyloxy, which can be substituted one to three times with C₁-C₈-alkyl, C₆-C₂₄aryloxy, which can be substituted one to three times with C₁-C₈-alkyl, or C₁-C₁₈alkoxy,
R² and R³ are independently of each other H, C₁-C₁₈alkyl, 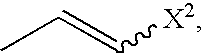
wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, Y stands for —CO—NR⁸R⁹, wherein
R⁸ and R⁹ are independently of each other hydrogen, C₁-C₂₅alkyl, or C₆-C₂₄aryl which can be substituted one to three times with C₁-C₈-alkyl, C₇-C₂₄aralkyl, which can be substituted one to three times with C₁-C₈-alkyl; or R⁸ and R⁹ together are C₂-C₈alkylene optionally substituted by C₁-C₈alkyl and/or optionally interrupted by —O—, —S— or —NR¹⁶³ or 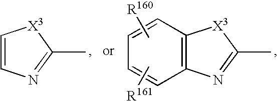
wherein X³ is O, S, or NR¹⁶³, wherein R¹⁶³ is a hydrogen atom, a C₁-C₂₅alkyl group, which is optionally interrupted by —O—, a cycloalkyl group, an aralkyl group, an aryl group, or a heterocyclic group, and
R¹⁶⁰ and R¹⁶¹ may be the same or different and are selected from hydrogen, C₁-C₂₅alkyl, cycloalkyl, aralkyl, alkenyl, cycloalkenyl, alkynyl, hydroxyl, a mercapto group, alkoxy, alkylthio, an aryl ether group, an aryl thioether group, aryl, a heterocyclic group, halogen, haloalkyl, haloalkenyl, haloalkynyl, a cyano group, an aldehyde group, a carbonyl group, a carboxyl group, an ester group, a carbamoyl group, a group NR¹²⁷R¹²⁸, wherein R¹²⁷ and R¹²⁸ independently of each other stand for a hydrogen atom, an alkyl group, an optionally substituted cycloalkyl group, an optionally substituted aryl group, an optionally substituted heteroaryl group, an optionally substituted heterocyclic group, an aralkyl group, or R¹²⁷ and R¹²⁸ together with the nitrogen atom to which they are bonded form a five or six membered heterocyclic ring, which can be condensed by one or two optionally substituted phenyl groups, a nitro group, a silyl group, a siloxanyl group, a substituted or unsubstituted vinyl group; or
R³ forms 5-, 6- or 7-membered rings with R¹, which can optionally be substituted by aryl, or phenyl, biphenyl, naphthyl, fluorenyl, anthryl, pyrenyl, phenanthryl, pyridyl, furanyl, benzofuranyl, triazinyl, or dibenzofuranyl, which can be substituted one to three times with C₁-C₈-alkyl; or which can be condensed one time by phenyl which can be substituted one to three times with C₁-C₄-alkyl; 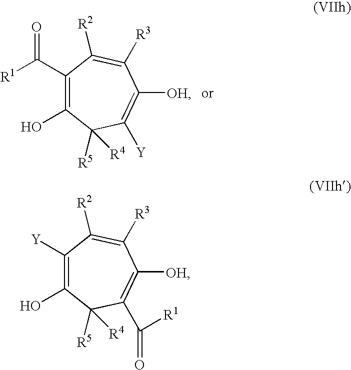
wherein Y and R¹ are as defined above,
R² and R³ are independently of each other H, C₁-C₁₈alkyl, 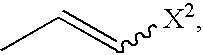
wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, or R² and R³ together form a ring and are a group 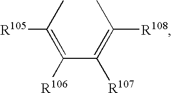
wherein R¹⁰⁵, R¹⁰⁶, R¹⁰⁷ and R¹⁰⁸ are independently of each other hydrogen, or C₁-C₁₈alkyl;
R⁴ and R⁵ are independently of each other hydrogen, C₁-C₂₅alkyl, which is optionally interrupted by —O—, or R⁴ and R⁵ together form a cyclohexane ring, which can optionally be substituted by C₁-C₈alkyl; or 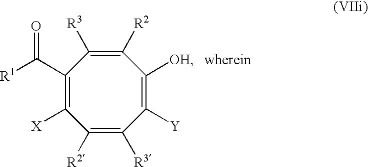
Y and R¹ are as defined above,
R², R³, R^{2′} and R^{3′} are independently of each other hydrogen, C₁-C₁₈alkyl, 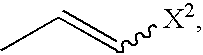
wherein X² is aryl, or heteroaryl, which can be substituted one to three times with C₁-C₈alkyl and/or C₁-C₈alkoxy; aryl or heteroaryl, which can be substituted one to three times with C₁-C₈alkyl and/or C₁-C₈alkoxy, or
R² and R³ together form a ring and are a group 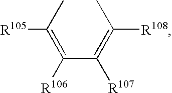
wherein R¹⁰⁵, R¹⁰⁶, R¹⁰⁷ and R¹⁰⁸ are independently of each other hydrogen, or C₁-C₁₈alkyl;
R⁴ and R⁵ are independently of each other hydrogen, C₁-C₂₅alkyl, which is optionally interrupted by —O—, or R⁴ and R⁵ together form a cyclohexane ring, which can optionally be substituted by C₁-C₈alkyl, with the proviso that at least one of R², R³, R^{2′} and R^{3′} are different from hydrogen.

7. A composition comprising a guest chromophore and a host chromophore, wherein the absorption spectrum of the guest chromophore overlaps with the fluorescence emission spectrum of the host chromophore, wherein the host chromophore is a fluorescent compound having a photoluminescence emission peak at 300 to 550 nm and wherein the guest chromophore is a compound of formula I according to claim 1.

8. A composition comprising to claim 7, wherein the host chromophore is represented by the formulae below 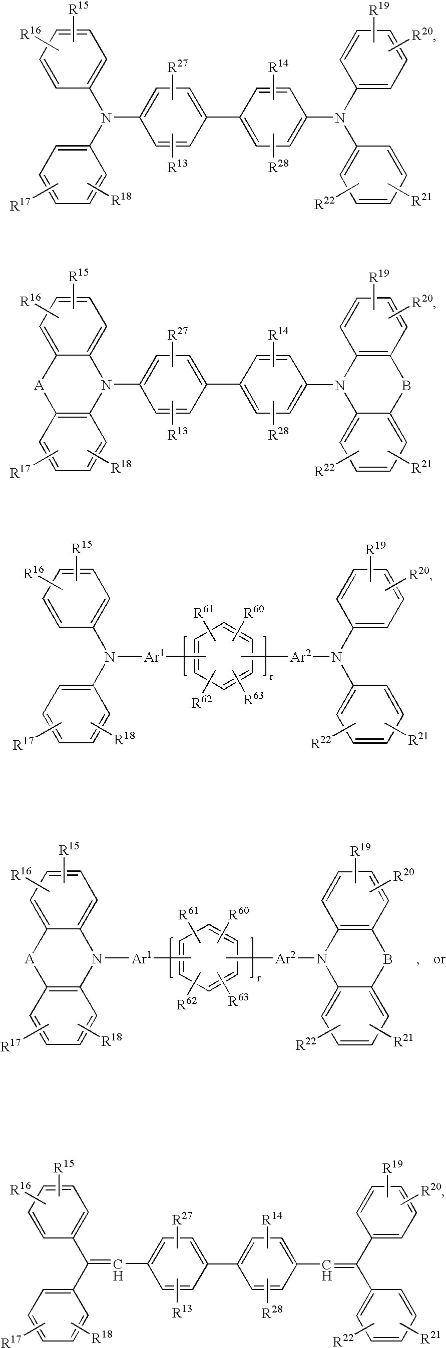
wherein r is an integer 1 to 10, R¹³, R¹⁴, R¹⁵, R¹⁶, R¹⁷, R¹⁸, R¹⁹, R²⁰, R²¹, R²², R²⁷, R²⁸, R⁶⁰, R⁶¹, R⁶², R⁶³ are independently of each other hydrogen, halogen, —CN, or C₁-C₁₂alkyl, which can optionally be substituted by one or more groups C₁-C₈alkyl or halogen; phenyl, naphthyl, or phenanthryl which can optionally be substituted by one or more groups C₁-C₈alkyl, halogen, —OR²³ or —NR²⁵R²⁶; —OR²³, —SR²⁴ or —NR²⁵R²⁶; wherein the substituents R²³, R²⁴, R²⁵, or R²⁶ optionally form 5-, 6- or 7-membered rings with adjacent substituents;
R²³ and R²⁴ are hydrogen, C₁-C₁₂alkyl or phenyl;
R²⁵ and R²¹ are independently of each other hydrogen, C₁-C₂₀alkyl; or R²⁵ and R²⁶ together are C₂-C₈alkylene optionally substituted by C₁-C₈alkyl and/or optionally interrupted by —O—, —S— or —N—,
A and B are independently of each other a direct bond, —CHR¹³—, —CHR¹³CHR¹⁴—, —NR²⁵—, —O—, or —S—,
Ar¹ and Ar² are identical or different from each other and are a C₆-C₂₄ aromatic or a C₁-C₂₄ hetero aromatic group, which can optionally be substituted by one or more groups C₁-C₈alkyl, halogen, —OR²³, —SR²⁴, or —NR²⁵R²⁶.

9. An EL device comprising a fluorescent compound according to claim 1.

10. A composition, comprising
(a) 0.01 to 50% weight, based on the total weight of a colored high molecular weight organic material, of a fluorescent material according to claim 1.
(b) 99.99 to 50% by weight, based on the total weight of the colored high molecular weight organic material, of a high molecular weight organic material, and
(c) optionally, customary additives in effective amounts.

11. (canceled)

12. A compound according to claim 1, wherein
when R¹ is C₆-C₂₄aryloxy it is phenoxy, when R¹ is C₇-C₂₄aralkyloxy it is benzyloxy, when R¹ is C₆-C₂₄thioaryloxy it is thiophenoxy, and when R¹ is C₇-C₂₄thioaralkyloxy it is thiobenzyloxy, each of which can optionally be substituted by one or more groups C₁-C₈alkyl, halogen, —CN, —OR⁶, —SR⁷ and/or —NR⁸R⁹;
and when either of R⁸ or R⁹ is C₇-C₂₄aralkyl it is phenyl-C₁-C₃alkyl and when either of R⁸ or R⁹ is C₆-C₂₄aryl it is phenyl or naphthyl, each of which can optionally be substituted by C₁-C₁₂alkyl, benzoyl, or C₁-C₁₂alkoxy.

13. A compound according to claim 2, wherein
R² and R³ are independently of each other phenyl, biphenyl, naphthyl, fluorenyl, anthryl, pyrenyl, phenanthryl, pyridyl, furanyl, benzofuranyl, triazinyl, or dibenzofuranyl, which can optionally be substituted by one or more groups C₁-C₈alkyl, phenyl, halogen, —OR⁶, —SR⁷ and/or —NR⁸R⁹.

14. A compound according to claim 3, wherein
A³ is phenyl or 1- or 2-naphthyl, which can be substituted one to three times with C₁-C₈alkyl and/or C₁-C₈alkoxy, and s stands for 0, 1, 2, 3 or 4.

15. A composition comprising a guest chromophore and a host chromophore according to claim 7, wherein the host chromophore is a fluorescent compound having a photoluminescence emission peak at 360 to 430 nm.

16. A composition according to claim 8, wherein
Ar¹ and Ar² are identical or different from each other and are selected from phenyl, naphthyl, and phenanthryl, each which can optionally be substituted by one or more groups C₁-C₈alkyl, halogen, —OR²³, —SR²⁴, or —NR²⁵R²⁶.

17. An EL device comprising a composition according to claim 7.

18. An EL device comprising a composition according to claim 8.

19. A composition, comprising
(a) 0.01 to 50% weight, based on the total weight of a colored high molecular weight organic material, a composition according to claim 7,
(b) 99.99 to 50% by weight, based on the total weight of the colored high molecular weight organic material, of a high molecular weight organic material, and
(c) optionally, customary additives in effective amounts.

20. A composition, comprising
(a) 0.01 to 50% weight, based on the total weight of a colored high molecular weight organic material, a composition according to claim 8,
(b) 99.99 to 50% by weight, based on the total weight of the colored high molecular weight organic material, of a high molecular weight organic material, and
(c) optionally, customary additives in effective amounts.
